(19) United States
(12) Patent Application Publication
(10) Pub. No.: US 2010/0016434 A1
(43) Pub. Date: Jan. 21, 2010

(54) Inhibitors of Egln3 Activity for the Treatment of Neurodegenerative Disorders

(76) Inventors: William G. Kaelin, JR., Boston, MA (US); Archana Bommi-Reddy, Boston, MA (US); Susanne Schlisio, Brookline, MA (US)

Correspondence Address: FIBROGEN, INC., 409 Illinois Street, San Francisco, CA, 94158, US

(21) Appl. No.: 11/988,534

(22) PCT Filed: Jul. 13, 2006
(86) PCT No.: PCT/US2006/027210

§ 371 Date: Jun. 15, 2009

Related U.S. Application Data
(60) Provisional application No. 60/698,879, filed on Jul. 13, 2005.

(51) Int. Cl. A61K 31/195; C12N 5/02; A61K 31/19

(52) U.S. Cl. 514/561; 435/375; 514/574

(57) ABSTRACT

The present invention provides methods for preventing or reducing neuronal apoptosis, particularly wherein the apoptosis is associated with a neurodegenerative disorder in a subject.

This application claims the benefit of U.S. Provisional Application Ser. No. 60/698,879, filed Jul. 13, 2005, incorporated by reference herein in its entirety.

### FIELD OF THE INVENTION

The present invention provides methods for preventing or reducing neuronal apoptosis, particularly wherein the apoptosis is associated with a neurodegenerative disorder in a subject. The invention also provides methods for increasing apoptosis in a subject having or at risk for having a cancer, particularly a cancer derived from neural crest cells.

### BACKGROUND

Normal embryonic development of the nervous system involves overproduction of neurons followed by a culling process. When an axonal projection from a neuron successfully reaches its target, various factors such as nerve growth factor (NGF) produced by the target tissue override an apoptotic signaling pathway and the neuron survives. Competing neurons, however, are deprived of these factors and subsequently undergo apoptosis. Failure to cull neurons can lead to overproduction of neuronal mass and may potentially result in cancers such as neuroblastoma and pheochromocytomas. Alternatively, excessive apoptosis of neurons, for example due to constitutive activation of the apoptotic pathway or loss of function in a component of the survival pathway, can result in various neurodegenerative conditions in infant and children.

Adult neurons may undergo a similar apoptotic process due to injury, toxic or hypoxic insult, or neurodegenerative disorders. In many of these disorders, neuronal apoptosis occurs due to a direct insult to the neuron, whereas in others the primary defect is in neuronal support cells such as glial cells. Whatever the underlying cause, all neuronal apoptotic events share common features and involve a prescribed set of factors that include c-Jun N-terminal kinase (JNK), which activates c-Jun and thereby increases transcription of various apoptotic factors. Additionally, the intrinsic mitochondrial death pathway involving Bax, Apaf-1, caspase 9, and caspase 3 has been shown to be critical for apoptosis in both developing and mature injured neurons. Bax, a bcl-2 family protein, is of particular importance in neuronal apoptosis. When overexpressed, Bax promotes cell death (Oltvai et al. (1993) Cell 74:6009-619), and Bax deficient sympathetic neurons deprived of NGF do not undergo normal apoptosis (Deckwerth et al. (1996) Neuron 17:401-411). Other Bcl-2 family members are also important in modulating neuronal apoptosis, e.g. phosphorylation of Bim by JNK potentiates Bax-dependent apoptosis. (See, e.g., Putcha et al. (2001) Neuron 38):899-914.)

Inappropriate neuronal apoptosis can lead to dementia and a decrease in motor control. Current treatment is limited, and improved methods that either delay or prevent neuronal loss would be particularly beneficial to those suffering from neuronal injury or neurodegenerative disease. The present invention provides methods of reducing or delaying neuronal apoptosis, thereby delaying or preventing loss of neuronal function in subjects with neurodegenerative disorders.

### SUMMARY OF THE INVENTION

A method for treating neuronal disorders, e.g., disorders characterized by undesirable neuronal apoptosis (e.g., neurodegenerative disorders) is described. The method entails administering an effective amount of an inhibitor of EGLN3. Therefore, in one embodiment, the invention provides a method for reducing apoptosis in a cell associated with or derived from the nervous system, the method comprising administering an inhibitor of EGLN3 enzyme activity to the cell. In one aspect, the administering is ex vivo. In another aspect, the administering is in vivo.

In one embodiment, the invention provides a method for reducing apoptosis associated with a neurodegenerative disorder in a subject. In one aspect, the disorder is associated with the central nervous system. In another aspect, the disorder is associated with the peripheral nervous system. In another aspect, the disorder involves both the central and peripheral nervous system.

In some embodiments the inhibitor is a small molecule. In one aspect, the inhibitor is an inhibitor of succinate dehydrogenase activity and may be selected from the group consisting of, but not limited to, malonic acid, 3-nitroproprionic acid, and theonyl trifluoracetone. In another aspect, the inhibitor is a 2-oxoglutarate analog. In a particular aspect, the 2-oxoglutarate analog is selected from the group consisting of dimethyloxalylglycine, N-oxalylglycine, N-oxalyl-2S-alanine, and N-oxalyl-2R-alanine. In various embodiments, the inhibitor can be administered alone or in combination with another agent for treating the neuronal disorder.

Among the disorders that might be treated with the current methods are Alzheimer's disease, Parkinson's disease, Huntington's disease, amyotrophic lateral sclerosis, multiple sclerosis, stroke, cerebral ischemia, AIDS-related dementia, neurodegeneration associated with bacterial infection, multi-infarct dementia, traumatic brain injury, spinal cord trauma, diabetic neuropathy and neurodegeneration associated with aging. Additionally, the methods may be used to retard or prevent neuronal apoptosis following injury or ischemic insult, e.g., stroke.

In another embodiment, the present invention provides a method comprising (a) identifying a patient suffering from or at risk for a neurodegenerative disorder; and (b) administering to the identified patient an inhibitor of EGLN3 enzyme activity.

In another embodiment, the present invention provides a method of increasing apoptosis in a subject by increasing EGLN3 levels or activity. The method comprises administering to the subject an agent that increases EGLN3 levels or activity. Such agents may include expression constructs encoding EGLN3 or an active fragment of EGLN3. Active fragments are those fragments that retain at least a portion of the hydroxylase activity of full-length EGLN3. Alternatively, the agent may be a compound that increases EGLN3 activity, e.g., a cofactor such as 2-oxoglutarate. In one embodiment, the method is used to increase apoptosis in a subject having or at risk for having a cell proliferative disorder. In one embodiment, the method is used to prevent or reduce growth of a tumor in the subject. In certain embodiments, the tumor is derived from neural crest cells. In particular embodiments, the tumor is selected from the group consisting of a melanoma, neuroblastoma, small cell lung carcinoma, and pheochromocytoma.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1: (A) Electrophorectic Mobility Shift Assay (EMSA) with ³²P-labeled DNA probes spanning Sp1 and AP1 sites in clusterin promoter and nuclear extracts prepared from 786-O VHL (−/−) renal carcinoma cells transfected to produce wild-type pVHL (WT8) or with empty vector (pRC3). WT=wild-type probe. ΔSp1=Sp1 site mutated probe. ΔAP1=API site mutated probe. Where indicated, unlabelled DNA containing a canonical SP1 or AP1 binding site was added as a competitor (COMP). Arrowhead=AP1 complex. (B and D) EMSA with ³²P-labelled AP1 site probe and nuclear extracts prepared from indicated cells lines. Anti-JunB antibody was added where indicated. Arrow=supershift complex. WTD10 and pRCB3 are A498 VHL(−/−) renal carcinoma cells transfected to produce wild-type pVHL or with empty vector, respectively. Panel (D) includes 786-O cells transfected to produce pVHL R64P, L119S, or L188V. (C) Immunoblot analysis of HeLa VHL (+/+) cervical carcinoma cells transfected with siRNA against VHL or scrambled siRNA. (E) Immunoblot analysis of nuclear extracts used in (D); (*)=non-specific band.

FIG. 2: (A) Immunoblot analysis of 786-O VHL(−/−) renal carcinoma cells retrovirally infected to produce the indicated short hairpin RNAs (shRNA) or wild-type pVHL (VHL). (B) Immunoblot analyis of RC3 cells and WT7 cells; where indicated, WT7 cells were treated with DFO or infected to produce HIF2α P405A; P531A or with empty retrovirus. (C) Immunoblot analysis of indicated 786-O subclones grown in presence or absence of DFO; (*)=non-specific band. (D) Immunoblot analysis of A498 and 786-O cells transfected to produce the indicated HA-pVHL variants or with empty vector. Arrow indicates a slowly migrating form of aPKC. (E) In vitro aPKC activity. Anti-aPKC immunoprecipitates of the indicated cell lines under antibody excess conditions were incubated with a peptidic aPKC substrate in the presence of ³²P-γ-ATP. Shown are incorporated ³²P values. (F) Immunoblot analysis of 786-O cells treated with the PKC inhibitor GF109230X.

FIG. 3: (A and B) Phase-contrast and fluorescent photomicrographs of PC12 cells transfected to produce GFP-Histone and grown in serum-rich media (‘undifferentiated) or serum-poor media supplemented with NGF for 10 days, which was then withdrawn for 24 hours. White arrows indicate apoptotic nuclei, which were quantitated in (B) as a percentage of GFP-positive nuclei. (C) Immunoblot analysis of PC12 grown in serum rich conditions (Undiff), serum poor conditions supplemented with NGF for 12 days, or after NGF withdrawal.

FIG. 4: (A and E) PC12 cells were transfected with a plasmid encoding GFP-Histone along with plasmid encoding wild-type JunB, dimerization-defective JunB (AbZip), c-RET, or the backbone plasmid (Empty). Shown are percent GFP-positive nuclei exhibiting apoptotic changes after growth in NGF for 5-7 days followed by NGF withdrawal (−). Control=cells transfected with GFP-His alone and maintained in NGF (+). Error bars=1 standard error. (B) PC12 cells were transfected with a plasmid encoding GFP-Histone along with siRNA against rat VHL (rVHL) and a plasmid encoding the indicated human pVHL variants. Where indicated rVHL siRNA was replaced with scrambled (SC) or luciferase (GL3) siRNA. Shown are percent GFP positive nuclei exhibiting apoptotic changes after growth in NGF for 5-7 days followed by NGF withdrawal (−). (C) Primary sympathetic neurons were electroporated to produce GFP alone (GFP) or GFP and Myc-tagged Jun B (JunB) and treated with NGF for 3 days. Shown are percent GFP-positive cells with apoptotic nuclei after continued NGF treatment (open bars) or 48 hours after NGF withdrawal (hatched bars). (D) Immunoblot of PC12 cells transfected to produce the indicated c-RET proteins or Myc-tagged PKCX and grown in the absence of NGF.

FIG. 5: (A) Anti-SM20/EGLN3 immunoblot analysis of PC12 cells treated as in FIG. 3A. SM-20 in vitro translate included in lane 1 as a control. (B and D) Representative fluorescent photomicrographs (B) and anti-HA immunoblot analysis (D) of PC12 cells transfected to produce GFP-Histone and the indicated HA-EGLN species. White arrows in (A) indicate apoptotic nuclei. (C) Percent of GFP-positive nuclei with apoptotic changes after transfection with 0.5 or 1.0 μg of the indicated plasmids. (E and F) Representative fluorescent photomicrographs of PC12 cells transfected with the indicated siRNAs and a plasmid encoding GFP-Histone followed by treatment with NGF for 10 days (‘+NGF’), which was then withdrawn for 24 hours (‘−NGF’). White arrows indicate apoptotic nuclei, which were quantitated in (E) as percent of GFP-positive nuclei. (G and H) Representative fluorescent photomicrographs (F) of PC12 cells transfected to produce GFP-Histone and treated with NGF for 5 days (‘+NGF’), which was then withdrawn for 24 hours (‘−NGF’). Where indicated cells were exposed to 100 μM CoCl₂ or 1% hypoxia during NGF withdrawal. White arrows indicate apoptotic nuclei, which were quantitated in (G) as % of GFP-positive nuclei.

FIG. 6: (A) Binding of ³⁵S-labeled pVHL to biotinylated HIF1α peptide after preincubation with unprogrammed reticulocyte lysate (RRL), EGLN3 in vitro translate (EGLN3 IVT), or EGLN3 IVT with the indicated concentrations of succinate and 2-oxoglutarate. ³⁵S-pVHL was loaded directly in lane 1 as a control. (B) FACS profiles of PC12 cells stained with the ROS-sensitive dye CM-H2DCFDA after treatment with the indicated SDH inhibitors or the ROS-inducing agent Rotenone (Ro; 20 μM) in the presence or absence of the ROS scavenger ascorbic acid (AA; 100 μM). C=control. (C and D) Representive fluorescent photomicrographs of PC12 cells (C) transfected to produce GFP-Histone alone (GFP-His) or GFP-Histone and EGLN3 (EGLN3). The SDH inhibitors 3-NPA (300 t-IM), MA (300 I-LM), or TTFA (200 ˜M) were added where indicated. White arrows indicate apoptotic nuclei, which were quantitated in (D) as percent of GFP-positive nuclei. (E) Immunoblot analysis of PC12 cells treated with the indicated chemicals or vehicle (C). (F) Percent of GFP-positive PC12 nuclei undergoing apoptosis after transfection to produce GFP-Histone alone (GFP-His) or GFP-Histone and EGLN3 (EGLN3) in the presence or absence of SDH inhibitors. 100 μM AA was also present where indicated. (G) Percent of GFP-positive PC 12 nuclei undergoing apoptosis after transfection with the indicated siRNAs and a plasmid encoding GFP-Histone followed by treatment with NGF for 5 days (‘+NGF’), which was then withdrawn for 24 hours (‘−NGF’). Where indicated 0.5 mM 2-oxoglutarate was added to the media 24 hours before NGF withdrawal.

FIG. 7: (A) Percent of GFP-positive PC12 nuclei undergoing apoptosis transfected to produce GFP-Histone alone (GFP-His) or GFP-Histone and EGLN3 (EGLN3) with or without Myc-tagged JunB. (B) Percent of GFP-positive PC12 nuclei undergoing apoptosis transfected with plasmids encoding GFP-Histone alone (GFP-His) or GFP-Histone and v-Jun (v-Jun) along with the indicated siRNAs. (C) Normalized luciferase values of PC12 cells transfected with reporter plasmid containing firefly luciferase under the control of EGLN3 promoter and plasmid encoding wild-type or mutant (DNA-binding defective) c-Jun. (D) Immunoblot analysis of PC12 cells infected with adenovirus encoding c-Jun or beta-galactosidase at indicated multiplicity of infection (MOI). (E and F) Immunoblot (E) and semi-quantitative RT-PCR analysis (F) of 786-O cells stably tranfected to produce the indicated pVHL species.

FIG. 8: Increased JunB Activity in pVHL-Defective Tumor Cells. EMSA with ³²P-labelled canonical API site probe and nuclear extracts prepared from indicated 786-O and A498 Subclones lines. WT8 and WTD10 are cells transfected to produce wild-type pVHL. PRC3 and pRCB3 are cells transfected with empty vector. NS=non-specific.

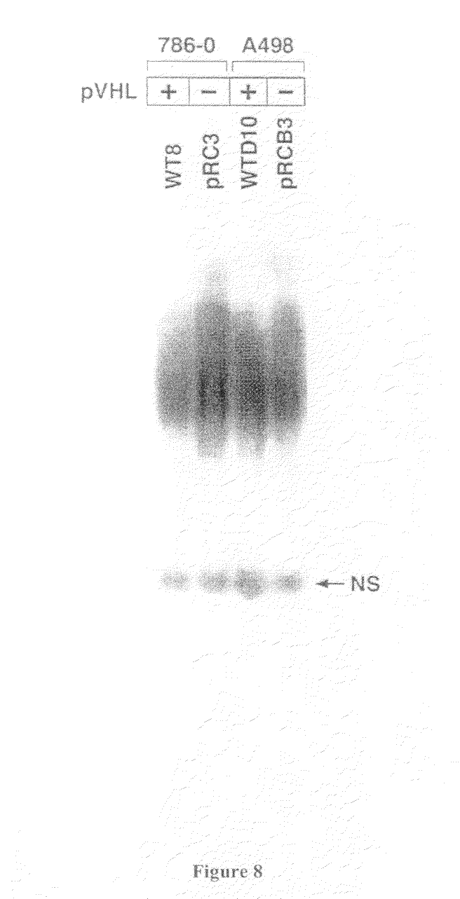

FIG. 9: JunB mRNA levels, normalized to TBP mRNA levels, for the indicated 786-O derivatives. First cDNA synthesis was carried out by First-Strand cDNA Synthesis kit (Amersham Biosciences Piscataway, N.J., USA) as described by the manufacturer's protocol. For real-time PCR, QuantiTect SYBR Green PCR kit (QIAGEN, Valencia, Calif., USA) and PCR amplification in GenAmp 5700 sequence detection system (Applied Biosystems, Foster City, Calif., USA) were used according to the manufacturer's protocol. Conditions for PCR were as follows; at 50° C. for 2 minutes, at 95° C. for 15 minutes, followed by 40 cycles at 94° C. for 15 seconds, 60° C. for 30 seconds, and 72° C. for 30 seconds. To verify that the used primer pair produced only a single band, a dissociation protocol was added after cycling, determining dissociation of the PCR products from 60 to 95. The assay included a no-template control, a standard curve of five serial dilution points (in steps of 10-fold) of a cDNA mixture, and each of the test cDNAs. TATA binding protein (TBP) was used as an internal control gene. The primer sequences were forward: GCACTAAAATGGAACAGCCCTT and reverse: CGGTTTCAGGAGTTTGTAGTCG for JUNB, and forward: CCCGAAACGCCGAATATAAT and reverse: CACACCATTTTCCCAGAACTGA for TBP.

FIG. 10: GF109203X does not affect HIF2α. (A and B) Immunoblot analysis of 786-O cells treated with the PKC inhibitor GF109230X.

FIG. 11: Inhibition of Neuronal Apoptosis by Jun B. Representative fluorescent photomicrographs of PC12 cells transfected to produce GFP-Histone and treated with NGF for 7 days (‘+NGF’), which was then withdrawn for 20 hours (‘−NGF”). Where indicated transfection mix contained a plasmid encoding wild-type JunB or dimerization-defective JunB. Arrows indicate apoptotic nuclei.

FIG. 12: EGLN3 H196A is hydroxylase-defective. (Left Panel). Autoradiogram of indicated ³⁵S-labelled in vitro translation products. (Right Panel). Binding of ³⁵S-labeled pVHL to biotinylated HIF1α peptide after preincubation with unprogrammed reticulocyte lysate (Mock) or indicated in vitro translation products. ³⁵S-pVHL was loaded directly in lane 1 as a control.

FIG. 13: (A) Percent of GFP-positive PC12 cells undergoing apoptosis transfected to produce GFP-Histone alone (−) or GFP-Histone and EGLN3 (+). Transfection mix also contained increasing amounts (0.5 or 1 μg) of plasmids encoding prolyl hydroxylation-defective HA-tagged HIF1α or HIF2α variants, where indicated by the triangles. (B and C) Firefly luciferase activity, normalized for renilla luciferase (B) and immunoblot analysis (C) of PC12 cells transfected as in (A) along with HIF response element (HRE) firefly luciferase reporter and SV40 promoter renilla luciferase reporter. The non-hydroxylatable HIF1α species is still relatively unstable and could not be detected with HA antibody (data not shown), which likely accounts for lower levels of firefly luciferase activity observed with HIF1α compared to HIF2α.

FIG. 14: Validation of SM20 siRNA. Immunoblot analysis of HeLa cervical carcinoma cells stably producing T7-tagged SM20 transfected with the indicated siRNAs.

FIG. 15: Validation of SDH D siRNA. Immunoblot analysis of HeLa cervical carcinoma cells stably producing Flag-tagged SDH D transfected with the indicated siRNAs.

FIG. 16: Representative fluorescent photomicrographs (A) of PC 12 cells transfected with the indicated siRNAs and a plasmid encoding GFP-Histone followed by treatment with NGF for 5 days (‘+NGF’), which was then withdrawn for 24 hours (‘−NGF’). White arrows indicate apoptotic nuclei, which were quantitated in (B) as % of GFP-positive nuclei.

FIG. 17: SDH inhibitors block Apoptosis after NGF withdrawal. % of GFP-positive PC12 nuclei undergoing apoptosis transfected to produce GFP-Histone and treated for NGF for 10 days (‘+’), which was then withdrawn for 24 hours (‘−’) in the presence or absence of the indicated SDH inhibitors (300 μM).

FIG. 18: Regulation of JunB by pVHL mutants. Immunoblot analysis of 786-O subclones producing the indicated pVHL mutants. PRC3=empty vector transfectants.

FIG. 19: Killing of SK-Mel-28 cells by EGLN3. Photomicrographs of SK-Mel-28 cells infected with Adenovirus encoding EGLN3 at indicated MOI. Hydroxylase inhibitor DMOG was added after infection where indicated.

### DESCRIPTION OF THE INVENTION

Before the present compositions and methods are described, it is to be understood that the invention is not limited to the particular methodologies, protocols, cell lines, assays, and reagents described, as these may vary. It is also to be understood that the terminology used herein is intended to describe particular embodiments of the present invention, and is in no way intended to limit the scope of the present invention as set forth in the appended claims.

It must be noted that as used herein and in the appended claims, the singular forms “a,” “an,” and “the” include plural references unless context clearly dictates otherwise. Thus, for example, a reference to “a fragment” includes a plurality of such fragments; a reference to a “compound” may be a reference to one or more compounds and to equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications cited herein are incorporated herein by reference in their entirety for the purpose of describing and disclosing the methodologies, reagents, and tools reported in the publications that might be used in connection with the invention. Nothing herein is to be construed as an admission that the invention is not entitled to antedate such disclosure by virtue of prior invention.

The practice of the present invention will employ, unless otherwise indicated, conventional methods of chemistry, biochemistry, molecular biology, cell biology, genetics, immunology and pharmacology, within the skill of the art. Such techniques are explained fully in the literature. See, e.g., Gennaro, A. R., ed. (1990) Remington's Pharmaceutical Sciences, 18th ed., Mack Publishing Co.; Hardman, J. G., Limbird, L. E., and Gilman, A. G., eds. (2001) The Pharmacological Basis of Therapeutics, 10th ed., McGraw-Hill Co.; Colowick, S. et al., eds., Methods In Enzymology, Academic Press, Inc.; Weir, D. M., and Blackwell, C. C., eds. (1986) Handbook of Experimental Immunology, Vols. I-IV, Blackwell Scientific Publications; Maniatis, T. et al., eds. (1989) Molecular Cloning: A Laboratory Manual, 2nd edition, Vols. I-III, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press; Newton, C. R., and Graham, A., eds. (1997) PCR (Introduction to Biotechniques Series), 2nd ed., Springer Verlag.

### DETAILED DESCRIPTION

The present invention provides methods for treating neuronal disorders, particularly those disorders characterized by undesirable neuronal apoptosis including, but not limited to, stroke, epilepsy, and neurodegenerative disorders. The methods entail administering an effective amount of an inhibitor of EGLN3. As used herein, the term “EGLN3” refers to various proteins alternatively referred to as EGLN3, PHD3, HPH1, and SM-20. EGLN3 includes, but is not limited to, human EGLN3 (GenBank Accession No. CAC42511; Taylor, supra), mouse EGLN3 (GenBank Accession No. CAC42517), and rat SM-20 (GenBank Accession No. AAA19321). EGLN3 also includes any orthologous protein in a cell derived from another species, particularly a mammalian species.

In one embodiment, the invention provides a method for reducing apoptosis in a cell associated with or derived from the nervous system, the method comprising administering an inhibitor of EGLN3 enzyme activity to the cell. In one aspect, the administering is ex vivo. Cells may be obtained from any source, preferably from a neuronal tissue obtained from a mammal. Cells may be cultured according to standard practices known to those of skill in the art. In another aspect, the administering is in vivo. The subject for in vivo administration may be any suitable eukaryote, particularly a mammal. In particular embodiments, the subject is a human.

In one embodiment, the invention provides a method for reducing apoptosis associated with a neurodegenerative disorder in a subject, the method comprising administering an inhibitor of EGLN3 enzyme activity to the subject. In one aspect, the disorder is associated with the central nervous system. In another aspect, the disorder is associated with the peripheral nervous system. In another aspect, the disorder involves both the central and peripheral nervous system. In some embodiments, the disorder is due to an ischemic or toxic insult that results in increased neuronal apoptosis. In other embodiments, the disorder is a neurodegenerative disorder of known or unknown origin that leads to progressive loss of neuronal function. Among the disorders that may be treated with the current methods are Alzheimer's disease, Parkinson's disease, Huntington's disease, amyotrophic lateral sclerosis, multiple sclerosis, stroke, cerebral ischemia, AIDS-related dementia, neurodegeneration associated with bacterial infection, multi-infarct dementia, traumatic brain injury, spinal cord trauma, diabetic neuropathy and neurodegeneration associated with aging. Additionally, the methods may be used to retard or prevent neuronal apoptosis following injury or ischemic insult, e.g., stroke.

In another embodiment, the present invention provides a method comprising (a) identifying a patient suffering from or at risk for a neurodegenerative disorder; and (b) administering to the identified patient an inhibitor of EGLN3 enzyme activity.

In another embodiment, the present invention provides a method of increasing apoptosis in a subject by increasing EGLN3 levels or activity. The method comprises administering to the subject an agent that increases EGLN3 levels or activity. Such agents may include expression constructs encoding EGLN3 or an active fragment of EGLN3. Active fragments are those fragments that retain at least a portion of the hydroxylase activity of full-length EGLN3. Such constructs are generally within the skill in the art and may contain any promoter appropriate for expression in the target tissue. Exemplary constructs are provided in the examples below. Alternatively, the agent may be a compound that increases EGLN3 activity, e.g., a cofactor such as 2-oxoglutarate. In one embodiment, the method is used to increase apoptosis in a subject having or at risk for having a cell proliferative disorder. In one embodiment, the method is used to prevent or reduce growth of a tumor in the subject. In certain embodiments, the tumor is derived from neural crest cells. In particular embodiments, the tumor is selected from the group consisting of a melanoma, neuroblastoma, small cell lung carcinoma, and pheochromocytoma.

The terms “inhibitor of EGLN3 enzyme activity” and “EGLN3 inhibitor”, and as abbreviated the term “inhibitor”, are used interchangeably and refer to any agent that reduces an activity of the EGLN3 enzyme. For example, for purposes of measuring activity, the activity of EGLN3 on hydroxylation of one or more proline residues on the alpha subunit of hypoxia inducible factor (HIFα) may be measured in the presence and absence of a test agent. A decrease in hydroxylation of HIFα when agent is present compared to when agent is absent would be indicative of an EGLN3 inhibitor for purposes of the present invention. Conversely, the terms “activator of EGLN3” and “EGLN3 activator”, and as abbreviated the term “activator”, are used interchangeably and refer to any agent that increases expression or activity of the EGLN3 enzyme. Activity of EGLN3, for purposes of identifying activators, can be measured as described above.

In various embodiments, the inhibitor of EGLN3 enzyme activity is a small molecule. In one aspect, the inhibitor is an inhibitor of succinate dehydrogenase activity and may be selected from the group consisting of, but not limited to, malonic acid, 3-nitroproprionic acid, and theonyl trifluoracetone. In another aspect, the inhibitor is a 2-oxoglutarate analog. In a particular aspect, the 2-oxoglutarate analog is selected from the group consisting of dimethyloxalylglycine, N-oxalylglycine, N-oxalyl-2S-alanine, and N-oxalyl-2R-alanine. Additional compounds that may inhibit EGLN3 are described in, e.g., Majamaa et al. (1984) Eur J Biochem 138:239-245; Majamaa et al. (1985) Biochem J 229:127-133; Kivirikko, and Myllyharju (1998) Matrix Biol 16:357-368; Bickel et al. (1998) Hepatology 28:404-411; Friedman et al. (2000) Proc Natl Acad Sci USA 97:4736-4741; Franklin (1991) Biochem Soc Trans 19):812-815; and Franklin et al. (2001) Biochem J 353:333-338. Additionally, compounds that inhibit EGLN3 can be selected from those described in, e.g., International Publication Nos. WO 03/049686, WO 02/074981, WO 03/080566, and WO 2004/108681. Compounds for use in the present methods inhibit EGLN3 enzyme activity, and may additionally inhibit activity of related enzymes, e.g., EGLN2, FIH, etc. Preferred compounds selectively inhibit EGLN3, i.e., show greater inhibition of EGLN3 than of related enzymes. In various embodiments, the inhibitor can be administered alone or in combination with another agent for treating the neuronal disorder.

The examples provided below demonstrate that inhibitors of EGLN3 activity are useful for treating disorders associated with undesirable neuronal apoptosis, e.g., neurodegenerative disorders such as Aizheimer's disease, Parkinson's disease, Huntington's disease, amyotrophic lateral sclerosis, multiple sclerosis, stroke, cerebral ischemia, AIDS-related dementia, neurodegeneration associated with bacterial infection, multi-infarct dementia, traumatic brain injury, spinal cord trauma, diabetic neuropathy and neurodegeneration associated with aging. For examples, the examples show that induction of neuronal apoptosis may be dependent on hydroxylase activity, particularly EGLN3 activity, when NGF is limiting. In addition, studies described below demonstrate that this EGLN3 pro-apoptotic activity requires SDH activity and that this requirement is due to feedback inhibition of EGLN3 by succinate, a compound that is converted to fumarate by SDH.

Pheochromocytomas are adrenal medullary tumors comprised of chromaffin cells, which are derived from sympathetic neuronal progenitor cells. Germline mutations in either NF1, c-RET, succinate dehydrogenase subunit genes (SDH B, SDH C, SDH D), or VHL are the most frequent cause of familial pheochromocytoma and are also common in seemingly sporadic (non-syndromic) pheochromocytoma. (Maher and Eng (2002) Hum Mol Genet 11:2347-2354; Neumann et al. (2002) N Engl J Med 343:1459-1466.) In contrast, somatic mutations of these genes are rare in non-hereditary pheochromocytomas (Maher and Eng, supra), raising the possibility that their functions must be altered during early development for pheochromocytomas to ensue. During embryogenesis most sympathetic neuronal precursor cells undergo c-Jun-dependent apoptosis as growth factors such as NGF become limiting. (Estus et al. (1994) J Cell Biol 127:1717-1727; Ham et al. (1995) Neuron 14:927-939; Schlingensiepen et al. (1994) Cell Mol Neurobiol 14:487-505; Xia et al. (1995) Science 270:1326-1331.) Disease-associated NF1 and c-RET mutations are known or suspected to enhance signaling by NGF receptors and promote neuronal survival. (Dechant (2002) Neuron 33:156-158; Vogel et al. (1995) Cell 82:733-742.) EGLN3 is induced in sympathetic neurons after NGF withdrawal and provokes apoptosis when overexpressed in pheochromocytoma cells. (Lipscomb et al. (1999) J Neurochem 73:429-432; Lipscomb et al. (2001) J Biol Chem 276:11775-11782; Straub et al. (2003) J Neurochem 85:318-328.)

The examples provided herein demonstrate that (1) EGLN3, but not EGLN1, is sufficient to induce neuronal apoptosis and does so in a hydroxylase-dependent manner; (2) EGLN3 acts downstream of c-Jun and is necessary for apoptosis after NGF withdrawal; and (3) SDH inactivation blocks neuronal apoptosis induced by EGLN3 overproduction or NGF withdrawal. Therefore, EGLN3 activity is both necessary and sufficient for the induction of apoptosis, and inhibition of EGLN3 activity, e.g., by inhibiting SDH, can reduce or prevent neuronal apoptosis and thereby reduce or prevent neuronal loss, e.g., due to neurodegenerative disorders.

### Inhibitors of EGLN3

The examples described herein provide mechanistic links between SDH mutations, EGLN3 activity, and escape from neuronal apoptosis. Inhibition of EGLN3 after SDH inactivation appears to be due to the accumulation of succinate, which can be transported to the cytosol by the dicarboxylate carrier located on the inner mitochondrial membrane. Thus, succinate dehydrogenase inhibitors including, but not limited to, malonic acid, 3-nitroproprionic acid, and theonyl trifluoracetone, can be utilized in the present methods to inhibit EGLN3 enzyme activity.

Additionally, small molecule compounds which may be used in the present methods include 2-oxoglutarate analogs including, but not limited to, dimethyloxalylglycine, N-oxalylglycine, N-oxalyl-2S-alanine, N-oxalyl-2R-alanine, an enantiomer of N-oxalyl-2S-alanine. Other N-oxalyl-amino acid compounds are among the potentially useful inhibitors.

Additional compounds that may be used to inhibit EGLN3 are described in, e.g., Majamaa et al. (1984) Eur J Biochem 138:239-245; Majamaa et al. (1985) Biochem J 229:127-133; Kivirikko, and Myllyharju (1998) Matrix Biol 16:357-368; Bickel et al. (1998) Hepatology 28:404-411; Friedman et al. (2000) Proc Natl Acad Sci USA 97:4736-4741; Franklin (1991) Biochem Soc Trans 19):812-815; and Franklin et al. (2001) Biochem J 353:333-338. Additionally, compounds that inhibit EGLN3 can be selected from those described in, e.g., International Publication Nos. WO 03/049686, WO 02/074981, WO 03/080566, and WO 2004/108681.

Additional inhibitors of EGLN3 enzyme activity may be identified using various methods known to those of skill in the art. For example, a screening assay as described in International Publication No. WO 2005/118836 may be used to screen compounds for selective activity against EGLN3. Compounds which may be screened using the assay may be natural or synthetic chemical compounds. Extracts of plants, microbes, or other organisms, which contain several characterized or uncharacterized components may also be used. Combinatorial libraries (including solid phase synthesis and parallel synthesis methodologies) provide an efficient way of testing larges numbers of different substances for ability to modulate hydroxylation. Further, the compounds described above can be similarly tested in various assays to identify those having particular selectivity for EGLN3. Such compounds are particularly advantageous in the present methods to reduce potential undesirable side effects.

### Formulation and Administration of Therapeutic Agents

The inhibitors of EGLN3 enzyme activity can be used alone or in combination with other compounds used to treat various neurodegenerative disorders. Combination therapies are useful in a variety of situations, including where an effective dose of one or more of the agents used in the combination therapy is associated with undesirable toxicity or side effects when not used in combination. This is because a combination therapy can be used to reduce the required dosage or duration of administration of the individual agents.

Combination therapy can be achieved by administering two or more agents, each of which is formulated and administered separately, or by administering two or more agents in a single formulation. Other combinations are also encompassed by combination therapy. For example, two agents can be formulated together and administered in conjunction with a separate formulation containing a third agent. While the two or more agents in the combination therapy can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or 10 weeks of each other. In some cases even longer intervals are possible. While in many cases it is desirable that the two or more agents used in a combination therapy be present within the patient's body at the same time, this need not be so.

Combination therapy can also include two or more administrations of one or more of the agents used in the combination. For example, if agent X and agent Y are used in a combination, one could administer them sequentially in any combination one or more times, e.g., in the order X-Y-X, X-X-Y, Y-X-Y, Y-Y-X, X-X-Y-Y, etc.

The inhibitor of EGLN3 enzyme activity, alone or in combination, can be combined with any pharmaceutically acceptable carrier or medium. Thus, they can be combined with materials that do not produce an adverse, allergic or otherwise unwanted reaction when administered to a patient. The carriers or mediums used can include solvents, dispersants, coatings, absorption promoting agents, controlled release agents, and one or more inert excipients (which include starches, polyols, granulating agents, microcrystalline cellulose, diluents, lubricants, binders, disintegrating agents, and the like), etc. If desired, tablet dosages of the disclosed compositions may be coated by standard aqueous or nonaqueous techniques.

The inhibitor of EGLN3 enzyme activity can be in the form of a pharmaceutically acceptable salt. Such salts are prepared from pharmaceutically acceptable non-toxic bases including inorganic bases and organic bases. Examples of salts derived from inorganic bases include aluminum, ammonium, calcium, copper, ferric, ferrous, lithium, magnesium, manganic salts, manganous, potassium, sodium, zinc, and the like. In some embodiments, the salt can be an ammonium, calcium, magnesium, potassium, or sodium salt. Examples of salts derived from pharmaceutically acceptable organic non-toxic bases include salts of primary, secondary, and tertiary amines, benethamine, N,N′-dibenzylethylenediamine, diethylamine, 2-diethylaminoethanol, 2-dimethylaminoethanol, diethanolamine, ethanolamine, ethylenediamine, N-ethylmorpholine, N-ethylpiperidine, epolamine, glucamine, glucosamine, histidine, hydrabamine, isopropylamine, lysine, methylglucamine, meglumine, morpholine, piperazine, piperidine, polyamine resins, procaine, purines, theobromine, triethylamine, trimethylamine, tripropylamine, and trolamine, tromethamine. Examples of other salts include arecoline, arginine, barium, betaine, bismuth, chloroprocaine, choline, clemizole, deanol, imidazole, and morpholineethanol. In one embodiment, salts are tris salts.

The inhibitor of EGLN3 enzyme activity can be administered orally, e.g., as a tablet or cachet containing a predetermined amount of the active ingredient, pellet, gel, paste, syrup, bolus, electuary, slurry, capsule; powder; granules; as a solution or a suspension in an aqueous liquid or a non-aqueous liquid; as an oil-in-water liquid emulsion or a water-in-oil liquid emulsion, via a liposomal formulation (see, e.g., EP 736299) or in some other form. Orally administered compositions can include binders, lubricants, inert diluents, lubricating, surface active or dispersing agents, flavoring agents, and humectants. Orally administered formulations such as tablets may optionally be coated or scored and may be formulated so as to provide sustained, delayed or controlled release of the active ingredient therein. The inhibitors can also be administered by captisol delivery technology, rectal suppository or parenterally.

The compositions may also optionally include other therapeutic ingredients, anti-caking agents, preservatives, sweetening agents, colorants, flavors, desiccants, plasticizers, dyes, and the like. The composition may contain other additives as needed, including for example lactose, glucose, fructose, galactose, trehalose, sucrose, maltose, raffinose, maltitol, melezitose, stachyose, lactitol, palatinite, starch, xylitol, mannitol, myoinositol, and the like, and hydrates thereof, and amino acids, for example alanine, glycine and betaine, and peptides and proteins, for example albumen. Examples of excipients for use as the pharmaceutically acceptable carriers and the pharmaceutically acceptable inert carriers and the aforementioned additional ingredients include, but are not limited to binders, fillers, disintegrants, lubricants, anti-microbial agents, and coating agents.

The EGLN3 inhibitors, either in their free form or as a salt, can be combined with a polymer such as polylactic-glycoloic acid (PLGA), poly-(I)-lactic-glycolic-tartaric acid (P(I)LGT) (WO 01/12233), polyglycolic acid (U.S. Pat. No. 3,773,919), polylactic acid (U.S. Pat, No. 4,767,628), poly(ε-caprolactone) and poly(alkylene oxide) (U.S. 2003/0068384) to create a sustained release formulation. Such formulations can be used to implants that release a compound of the invention or another agent over a period of a few days, a few weeks or several months depending on the polymer, the particle size of the polymer, and the size of the implant (see, e.g., U.S. Pat. No. 6,620,422).

The EGLN3 inhibitors can be administered, e.g., by intravenous injection, intramuscular injection, subcutaneous injection, intraperitoneal injection, topical, sublingual, intraarticular (in the joints), intradermal, buccal, ophthalmic (including intraocular), intranasaly (including using a cannula), or by other routes. The inhibitors can be administered orally, e.g., as a tablet or cachet containing a predetermined amount of the active ingredient, gel, pellet, paste, syrup, bolus, electuary, slurry, capsule, powder, granules, as a solution or a suspension in an aqueous liquid or a non-aqueous liquid, as an oil-in-water liquid emulsion or a water-in-oil liquid emulsion, via a micellar formulation (see, e.g. International Publication WO 97/11682) via a liposomal formulation (see, e.g., European Patent 736299, and International Publications WO 99/59550 and WO 97/13500), via formulations described in International Publication WO 03/094886, or in some other form. Orally administered compositions can include binders, lubricants, inert diluents, lubricating, surface active or dispersing agents, flavoring agents, and humectants. Orally administered formulations such as tablets may optionally be coated or scored and may be formulated so as to provide sustained, delayed or controlled release of the active ingredient therein.

The EGLN3 inhibitors can also be administered transdermally (i.e. via reservoir-type or matrix-type patches, microneedles, thermal poration, hypodermic needles, iontophoresis, electroporation, ultrasound or other forms of sonophoresis, jet injection, or a combination of any of the preceding methods (Prausnitz et al. (2004) Nature Rev Drug Discovery 3:115)). The agents can be administered using high-velocity transdermal particle injection techniques using the hydrogel particle formulation described in U.S. Patent Application Publication 2002/0061336. Additional particle formulations are described in International Publications WO 00/45792, WO 00/53160, and WO 02/19989. An example of a transdermal formulation containing plaster and the absorption promoter dimethylisosorbide can be found in International Publication WO 89/04179. International Publication WO 96/11705 provides formulations suitable for transdermal administration. The inhibitors can be administered in the form a suppository or by other vaginal or rectal means. The agents can be administered in a transmembrane formulation as described in International Publication WO 90/07923. The agents can be administered non-invasively via the dehydrated particles described in U.S. Pat. No. 6,485,706. The agent can be administered in an enteric-coated drug formulation as described in International Publication WO 02/49621. The agents can be administered intranasaly using the formulation described in U.S. Pat. No. 5,179,079. Formulations suitable for parenteral injection are described in International Publication WO 00/62759. The agents can be administered using the casein formulation described in U.S. Patent Application Publication 2003/0206939 and International Publication WO 00/06108. The agents can be administered using the particulate formulations described in U.S. Patent Application Publication 2002/0034536.

The inhibitors of EGLN3 enzyme activity, alone or in combination with other suitable components, can be administered by pulmonary route utilizing several techniques including but not limited to intratracheal instillation (delivery of solution into the lungs by syringe), intratracheal delivery of liposomes, insufflation (administration of powder formulation by syringe or any other similar device into the lungs) and aerosol inhalation. Aerosols (e.g., jet or ultrasonic nebulizers, metered-dose inhalers (MDIs), and dry-powder inhalers (DPIs)) can also be used in intranasal applications.

These and other embodiments of the present invention will readily occur to those of ordinary skill in the art in view of the disclosure herein and are specifically contemplated.

### Examples

The invention is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications fall within the scope of the appended claims.

The following methods and materials were employed in the examples described below.

### Cell Lines

The 786-O and A498 renal carcinoma cell line derivatives made by stable transfection or retroviral infection are described elsewhere (Kondo et al. (2003) Cancer Cell 1:237-246; Lonergan et al. (1998) Mol Cell Biol 18:732-741) and were maintained in DMEM containing 10% Fetal Clone (Hyclone, Logan Utah) and, where appropriate, G418 and/or puromycin, in the presence of 10% CO₂ at 37° C. Undifferentiated PC12 cells were maintained in DMEM containing 10% Fetal Bovine Serum (Hyclone) and 5% Horse Serum (Sigma-Aldrich, St. Louis Mo.) in 37° C., 10% C0₂ incubator.

### Plasmids

The human JunB open reading frame cDNA in a Gateway entry plasmid (a gift of Dr. Marc Vidal, Dana-Farber Cancer Institute) was transferred to the Gateway expression vector pDEST47 (Invitrogen Corp., Carlsbad Calif.) by recombination cloning to make pDEST47-JunB. The JunB cDNA was PCR amplified with primer A (5′-ggggacaagtttgtacaaaaaagcaggctatgtgcactaaaatggaacagccct-3′; SEQ ID NO:1) and primer B (5′-ggggaccactttgtacaagaaagctgggtcctagcgcgcgatgcgctccagctt-3′; SEQ ID NO:2) to make the JunBAbZip cDNA, which was transferred to pDEST47 by sequential BP and LR recombination reactions according to manufacturer's instructions (Invitrogen). The human c-RET cDNA, encoding the short 1072 residue c-RET isoform, in a Gateway entry plasmid (a gift of Marc Vidal) was similarly transferred to pDEST47 (Invitrogen) by recombination cloning to make pDEST47-c-RET. The c-RET cDNA was PCR amplified with primer C (5′-ccactgtgcgacgagctgcgccgcacggtgatcgcagcc-3′; SEQ ID NO:3) and primer D (5′-ggctgcgatcaccgtgcggcgcagctcgtcgcacagtgg-3′; SEQ ID NO:4) to make the constitutive active C634R c-RET mutant, which was also transferred to pDEST47.

The pVHL expression plasmids were as described by Hoffman et al. (2001; Hum Mol Genet 10:1019-1027.)

The expression plasmids for HA-EGLN1 (Ivan et al. (2002) Proc Natl Acad Sci USA 99:13459-13464) and HA-HIF2α P405A;P531A (Kondo et al. (2003) PLoS Biol 1:439-444) were described previously and the plasmids for HA-EGLN2, HA-EGLN3, and HA-HIF1α P402A;P564A were made analogously. HA-EGLN3 H196A was generated using site-directed mutagenesis kit (GENEEDITOR; Promega Corp., Madison Wis.). The pcDNA-Myc-JunB was made by PCR amplification of IMAGE-clone MGC 10557 with primers that introduced a 5′ BamHI site and 3′ EcoRI site followed by ligation into 5x-myc-pcDNA3. The plasmid encoding human Δc-Jun, which harbors mutations analogous to the chicken v-Jun mutations, was described before (Wei et al. (2005) Cancer Cell 8:25-33). To make the c-Jun leucine zipper mutant, a c-Jun cDNA corresponding to residues 1-255 was amplified by PCR with primers that introduced a 5′ BamHI site and 3′ EcoRI site and ligated into 5x-myc-pcDNA3. All cDNAs were sequence verified.

### EMSA

Synthetic oligonucleotides were end-labeled with [γ³²P] ATP and T4 DNA Kinase (New England Biolabs, Ipswich Mass.) according to the manufacturer's instructions and annealed in vitro for use in electophoretic mobility shift assays containing 5 μg of nuclear extract (9 μg for supershift assays), prepared using a NUCLEAR EXTRACT kit (Active Motif, Carlsbad Calif.), in a final volume of 20 μl in the presence of 10 mM Tris-HCI (pH7.5) 50 mM NaCI, 1 mM EDTA, 10% glycerol, 1 mM DTT and 2 μg of poly(dI-dC). The clusterin promoter-derived EMSA probe sequences (sense strands) were: WT 5′-ttctttgggcgtgagtcatgca-3′ (SEQ ID NO:5), ΔAP1 5′-ttctttgggcgtgaggcatgca-3′ (SEQ ID NO:6), ΔSp1 5′-ttctttgttcgtgagtcatgca-3′ (SEQ ID NO:7). Canonical binding site probe sequences (sense) were: Sp1 5′-attcgatcggggcggggcgagc-3′ (SEQ ID NO:8) and AP1 5′-cgcttgatgagtcagccggaa-3′ (SEQ ID NO:9). Competitor unlabeled probes were annealed in vitro and used at 50-fold molar excess of labeled probe. Supershift assays were performed with polyclonal anti-JunB NUSHIFT antibody (Active Motif) and NUSHIFT kit (Active Motif) according to the manufacturer's instructions. Differential supershifts were not observed with antisera against c-Fos or c-Jun (data not shown).

### Immunoblot Analysis

Twenty μg of nuclear extract per lane, prepared using a NE-PER extraction kit (Pierce Biotechnology, Inc., Rockford Ill.) and measured by the Bradford assay, was resolved on 10% or 12% SDS-PAGE gels and transferred to nitrocellulose membrane (Bio-Rad Laboratories, Hercules Calif.) to detect endogenous JunB and HIF2α. After blocking in TBS with 5% nonfat milk, the membranes were probed with anti-JunB monoclonal antibody (C-11; Santa Cruz Biotechnology, Inc., Santa Cruz Calif.) or anti-HIF2α rabbit polyclonal antibody (NB100-122; Novus Biologicals, Inc., Littleton Colo.). Bound protein was detected with a Horseradish Peroxidase (HRP)-conjugated secondary antibodies and an enhanced chemiluminesence kit (Pierce).

HA-EGLN1, HA-EGLN2, HA-EGLN3, HA-H196A and HA-HIF2α were detected in whole cell extracts using polyclonal α-HA (Y-11; Santa Cruz Biotechnology). HA-HIF1α was detected using monoclonal anti-HIF1α (BDB Transduction labs, Lexington Key.). The antibody against rat SM-20 was described previously (Straub et al. (2003) J Neurochem 85:318-328). The antibody against rodent HIF1α, which also recognizes HIF2α (data not shown), was described in Berra et al. (2003; EMBO J 22:4082-4090).

siRNA

Short interfering RNA (siRNA) oligonucleotides were purchased from Dharmacon. Sense strand sequences were: rVHL: 5′-aauguugauggacagccuauu-3′ (SEQ ID NO:10), hVHL #7: 5′-aauguugacggacagccuauu-3 (SEQ ID NO:11), GL3: 5′-cuuacgcugaguacuucgauu-3′ (SEQ ID NO:12), Scramble: 5′-aacagucgcguuugcgacugg-3′ (SEQ ID NO:13), SM20 #1: 5′-cagguuauguucgucaugu-dTdT (SEQ ID NO:14), SM20 #2: 5′-uucuccuggucagaccgca-dTdT (SEQ ID NO:15), SDHD #1: 5′-guugccaugcuguggaagc-dTdT (SEQ ID NO:16), SDHD #2: 5′-uuggacaagugguuacuga-dTdT (SEQ ID NO:17).

### In Vitro Kinase Assays

In vitro kinase assays were performed as described elsewhere (Standaert et al., 2004) using a rabbit polyclonal antibody that recognizes the C-termini of both PKC-λ and PKC-ζ (Santa Cruz Biotechnology). Immunoprecipitated aPKCs were incubated for 8 min at 30° C. in 100 μl buffer containing 50 mM Tris/HCl (pH,7.5), 100 μM Na₃V0₄, 100 μM Na₄P₂O₇, 1 mM NaF, 100 μM PMSF, 4 μg phosphatidylserine (Sigma-Aldrich), 50 μM [γ-³²P]ATP (PerkinElmer Life And Analytical Sciences, Inc., Wellesley Mass.), 5 mM MgCl₂ and, as substrate, 40 μM serine analogue of the PKC-ε pseudosubstrate (Invitrogen). After incubation, ³²P-labeled substrate was trapped on P-81 filter papers and counted.

### Apoptosis Assays

Undifferentiated PC12 cells were plated onto collagen-coated 6-well plates 1 day before transfection with LIPOFECTAMINE 2000 reagent (Invitrogen) according to the manufacturer's instructions. Transfection mixes contained 500 ng of a plasmid encoding GFP-Histone (a gift of Dr. Geoffrey M. Wahl, The Salk Institute for Biological Studies), 1 μg of the cDNA expression plasmid of interest and, where indicated, 100 nM of siRNA. 48 hours later the cells were trypsinized, transferred to collagen-coated p100 dishes, and grown in Dulbecco's modified Eagle's medium (DMEM) supplemented with 1% horse serum and NGF (50 ng/ml) for 5-7 days. For NGF withdrawal cells were washed once with serum-free medium followed by incubation in NGF-free medium containing a neutralizing antibody against the 2.5S and 7S forms of NGF (Accurate Chemical & Scientific Corp., Westbury N.Y.) at a 1:400 dilution. Control cells were washed once in NGF-free medium and then returned to NGF-containing medium. Nuclei that were condensed or fragmented were scored as apoptotic. Approximately 400 cells were scored for each set of conditions and all assays were performed in triplicate.

### Isolation of Primary Sympathetic Neurons

Sympathetic neurons were isolated from the superior cervical ganglia (SCG) as described by Palmada et al. (2002; J Cell Biol 158:453-461). Briefly, SCG from Sprague-Dawley rats were isolated at postnatal day 4, and sympathetic neurons were dissociated with 0.25% trypsin and 0.3% collagenase for 30 min at 37° C. After dissociation, the neurons were electroporated with pmax-GFP alone (Amaxa, Inc., Gaithersburg Md.) or pmax-GFP along with JunB expression plasmid according to the manufacturer's instructions (rat neuron NUCLEOFECTOR kit; Amaxa). The neurons were then cultured on poly-L-ornithine and laminin coated 4 well slides (Nalge Nunc International, Rochester N.Y.) in ULTRACULTURE medium (BioWhittaker, Inc., Walkersville Md.) supplemented with 3% fetal calf serum (Invitrogen), 2 mM L-glutamine (Invitrogen), and 20 ng/ml NGF (Harlan, Indianapolis Ind.). The neurons were maintained for 3 days in the presence of NGF and then washed twice in ULTRACULTURE medium lacking NGF, once with ULTRACULTURE containing an antibody to NGF at 0.1 μg/ml (Chemicon International, Temecula Calif.), and returned to NGF-free media. The cells were fixed in paraformaldehyde 48 hours later and the number of GFP positive neurons with apoptotic nuclei, identified by DAPI staining (Vector Laboratories, Burlingame Calif.), were counted. At least 75 neurons were evaluated for each condition.

### Hydroxylation Assays

Hydroxylation assays were performed essentially as described by Ivan et al. (2002; Proc Natl Acad Sci USA 99:13459-13464).

### ROS Analysis

PC12 cells were incubated for 1 hr with 5 μM CM-H2DCFDA (Molecular Probes), harvested, resuspended at 106 cells/ml in PBS supplemented with 7% FBS, and analyzed by FACS.

### Luciferase Assays

The HRE reporter was described in Kondo et al. (2002; Cancer Cell 1:237-246). The SM20 promoter reporter was described in Menzies et al. (2004; Biochem Biophys Res Commun 317:801-810). Luciferase assays were performed in triplicate using a luciferase dual reporter assay system (Promega).

### Adenovirus

The Adenovirus encoding c-Jun was described by Yu et al. (2001; Circulation 104:1557-1563).

### Semiguantitative RT-PCR

Total RNAs were extracted with RNeasy Mini Kit (Qiagen, Inc., Valencia Calif.). cDNA synthesis and PCR amplification were performed with Superscript One-Step RT-PCR (Invitrogen) using 1 μg total RNA. EGLN3 cDNA was amplified with sense primer (5′-gcgtctccaagcgaca; SEQ ID NO: 18) and antisense primer (5′-gtcttcagtgagggcaga; SEQ ID NO: 19) for 32 cycles. As a control, GAPDH cDNA was also amplified with sense primer (5′-ctacactgagcaccaggtggtctc; SEQ ID NO:20) and antisense primer (5′-gatggatacatgacaaggtgcggc; SEQ ID NO:21). 10 μl aliquots of the PCR reaction (50 μl) were separated on a 2% agarose gel.

### Example 1

### pVHL Downregulates JunB

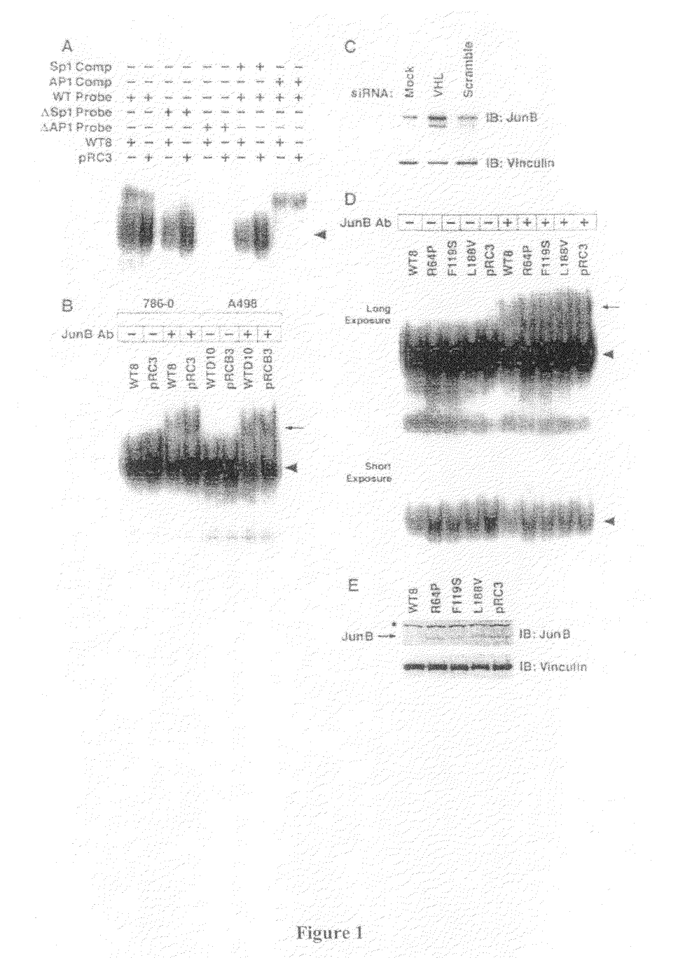

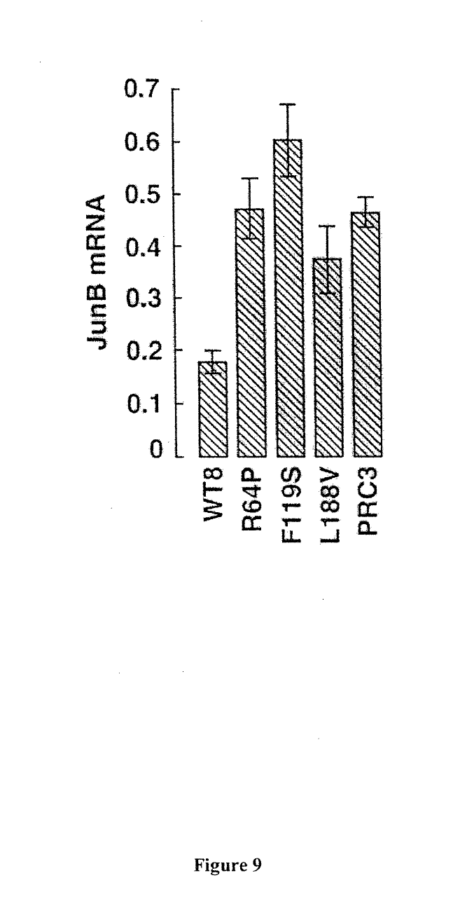

The von Hippel Lindau protein (pVHL) is part of an E3 ubiquitin ligase complex that targets proteins, particularly the alpha subunit of the heterodimeric transcription factor HIF (hypoxia-inducible factor), for degradation. Mutations in pVHL result in abnormal growth of blood vessels in various organs, and can lead to hemangioblastomas and renal cell carcinomas. Certain mutations in pVHL, referred to as Type 2 pVHL mutants, are associated with a high incidence of pheochromocytomas. Although most mutations in pVHL lead to increased levels of HIF, Type 2C pVHL mutants show normal regulation of HIF. Although HIF is the most well characterized target, other proteins appear to be regulated by pVHL. For example, although mRNA levels for the secreted protein clusterin were attenuated in VHL (−/−) renal carcinoma cells, clusterin did not behave like a HIF target and Type 2C pVHL mutants, in contrast to wild-type pVHL, did not restore clusterin expression when reintroduced into such cells. The clusterin promoter contains binding sites for Myb, AP-1, and Sp1. (Cervellera et al. (2000) J Biol Chem 275:21055-21060; Herault et al. (1992); Nucleic Acids Res 20:6377-6383; Jin and Howe (1997) J Biol Chem 272:26620-26626.) We found that wild-type, but not mutant, pVHL activated luciferase reporter plasmids containing the clusterin promoter unless the AP-1 site was destroyed (data not shown). This led us to examine the status of specific AP-1 family members in cells that do or do not contain wild-type pVHL. In electrophoretic mobility shift assays (EMSA) we detected increased API activity (FIG. 1A and FIG. 9), due at least partly to JunB (FIG. 1B), in renal carcinoma cells lacking wild-type pVHL. This effect was specific to JunB because the AP-1 family members c-Jun and c-Fos were not affected by pVHL in these assays (data not shown). Jun B protein levels were also elevated in HeLa cervical carcinoma cells after elimination of pVHL with 3 independent siRNAs (FIG. 1C and data not shown).

### Example 2

### Regulation of JunB by pVHL Involves Both Atypical Protein Kinase C and HIF

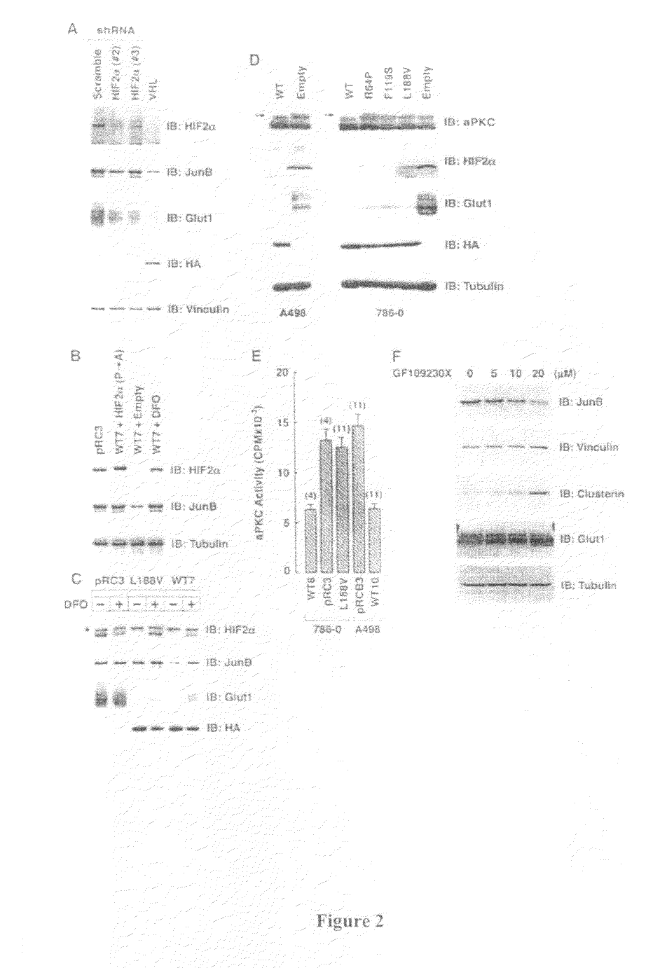

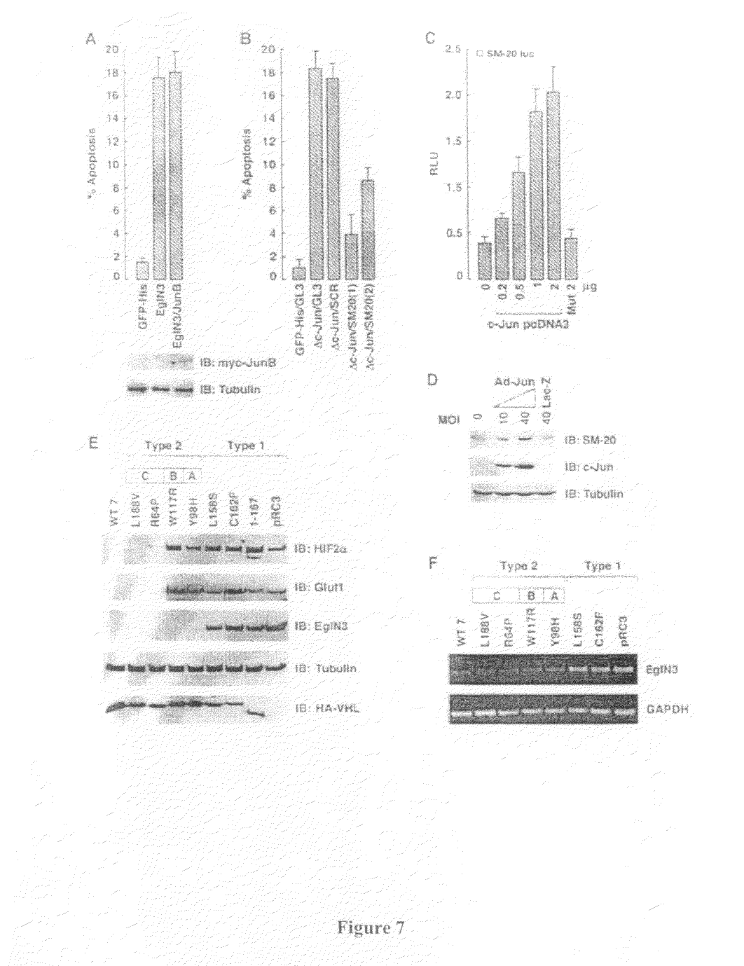

786-O VHL (−/−) cells produce HIF2α but not HIF1α. (Maxwell et al. (1999) Nature 399:271-275.) Type 2C pVHL mutants normalize HIF2α levels when reintroduced into 786-O cells (Clifford et al. (2001) Hum Mol Genet 10:1029-1038; Hoffman et al. (2001) Hum Mol Genet 10:1019-1027) (see also FIGS. 2C and 7E), but did not normalize JunB levels (FIGS. 1D and E). Similarly, downregulation of HIF2α in 786-O cells with short hairpin RNAs (shRNA) had little or no effect on JunB levels, in contrast to canonical HIF targets such as GLUT1 (FIG. 2A). On the other hand, JunB was induced in cells containing wild-type pVHL that were engineered to produce a stabilized form of HIF2α or treated with the hypoxia mimetic deferoxamine (DFO) (FIG. 2B). Collectively, these results suggested that either JunB is already maximally stimulated at the residual HIF levels achieved with Type2C mutants or HIF2α shRNA, or that regulation of JunB by pVHL involves HIF-dependent and HIF-independent pathways. The former possibility seems unlikely because JunB was not induced by DFO in cells producing the Type 2C pVHL mutant L188V, but was induced in cells producing wild-type pVHL, even though these cells produced nearly identical levels of HIF2α and GLUT1 (FIG. 2C).

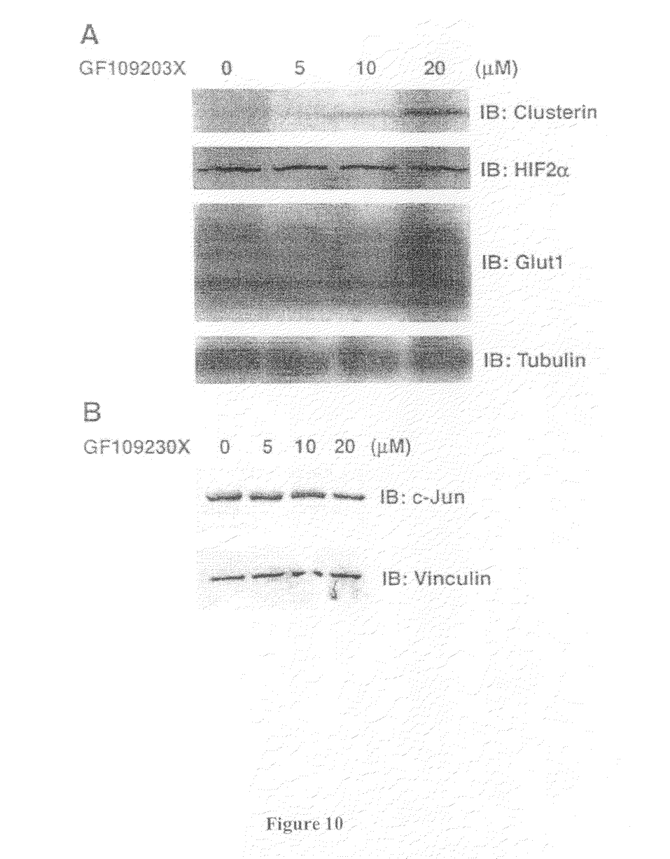

The increased JunB protein levels observed in pVHL-defective cells, including those producing Type 2C mutants, was associated with an ˜2-3 fold increase in JunB mRNA levels (FIG. 9). Transcription of JunB is regulated by atypical PKC family members (aPKC) (Kieser et al. (1996) Genes Dev 10:1455-1466) and pVHL has been reported to polyubiquitinate aPKC (Okuda et al. (2001) J Biol Chem 276:43611-43617). In this earlier report, however, aPKC protein levels were not increased in pVHL-defective cells, leading the authors to speculate that pVHL targets a minor subpopulation of aPKC corresponding to the hyperphosphorylated, activated, form of the enzyme. In support of this idea, we detected an increase in a slowly migrating form of aPKC in immunoblot assays of pVHL-defective cells (FIG. 2D). This aPKC species is likely to be a phosphorylated, and hence presumably activated, form of aPKC because a single, faster migrating, aPKC band was detected after treatment with lambda phosphatase (data not shown). Importantly, this slowly migrating aPKC species was suppressed by wild-type pVHL, but not Type 2C pVHL mutants, and its abundance was mirrored by changes in aPKC kinase activity (FIG. 2E). JunB, in contrast to HIF2α and the AP1 family member c-Jun, was also downregulated with a pharmacological aPKC inhibitor (FIG. 2F and FIG. 10). These results suggest that pVHL regulates JunB via both aPKC and by HIF.

### Example 3

### Loss of pVHL Promotes Neuronal Survival

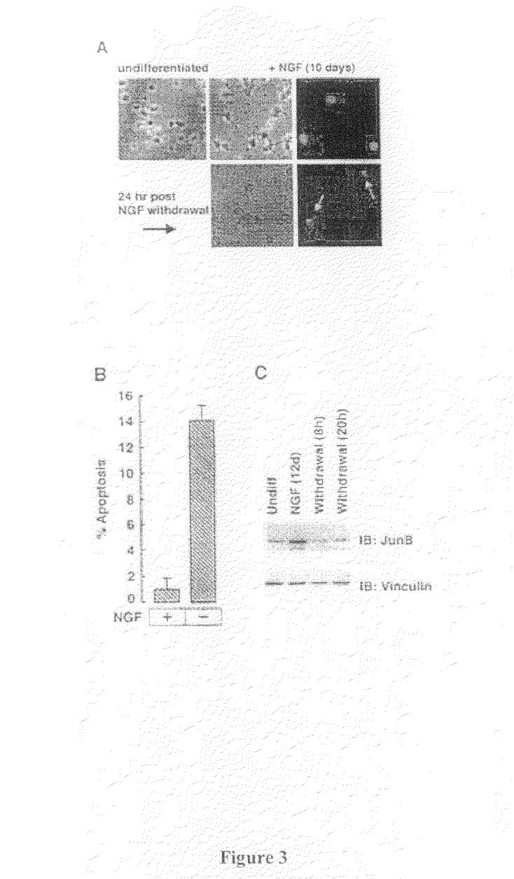

Pheochromocytoma cells are derived from sympathetic neuronal precursor cells and PC12 rat pheochromocytoma cells, which are VHL +/+, have been used as a model to study the regulation of neuronal survival by Nerve Growth Factor (NGF). During normal neuronal development many cells undergo apoptosis as they compete for NGF. Loss of NGF leads to activation of c-Jun and the induction of apoptosis. (Ham et al. (1995) Neuron 14:927-939; Palmada et al. (2002) J Cell Biol 158:453-461; Schlingensiepen et al. (1994) Cell Mol Neurobiol 14:487-505; Xia et al. (1995) Science 270:1326-1331.) PC12 cells resemble differentiated sympathetic neurons when grown under low serum conditions in the presence of NGF, displaying plasma membrane ruffling, cellular flattening and enlargement, and formation of stable neurites (Greene (1978) J Cell Biol 78:747-755; Greene and Tischler (1976) Proc Natl Acad Sci USA 73:2424-2428) (FIG. 3A). The nuclei of PC12 cells transfected to produce GFP-histone and induced to differentiate with NGF were uniform and intact. Consistent with earlier studies, NGF withdrawal led to morphological changes characteristic of apoptosis including plasma membrane blebbing, cell body shrinkage, neurite retraction, and nuclear condensation and fragmentation (Deckwerth and Johnson (1993) J Cell Biol 123:1207-1222; Edwards and Tolkovsky (1994) J Cell Biol 124:537-546) (FIGS. 3A and B). The percentage of apoptotic cells at any time point is <20% with this experimental paradigm because PC12 cells do not die synchronously under these conditions. (Francois et al. (2001) Mol Cell Neurosci 18:347-362; Messam and Pittman (1998) Exp Cell Res 238-389-398.)

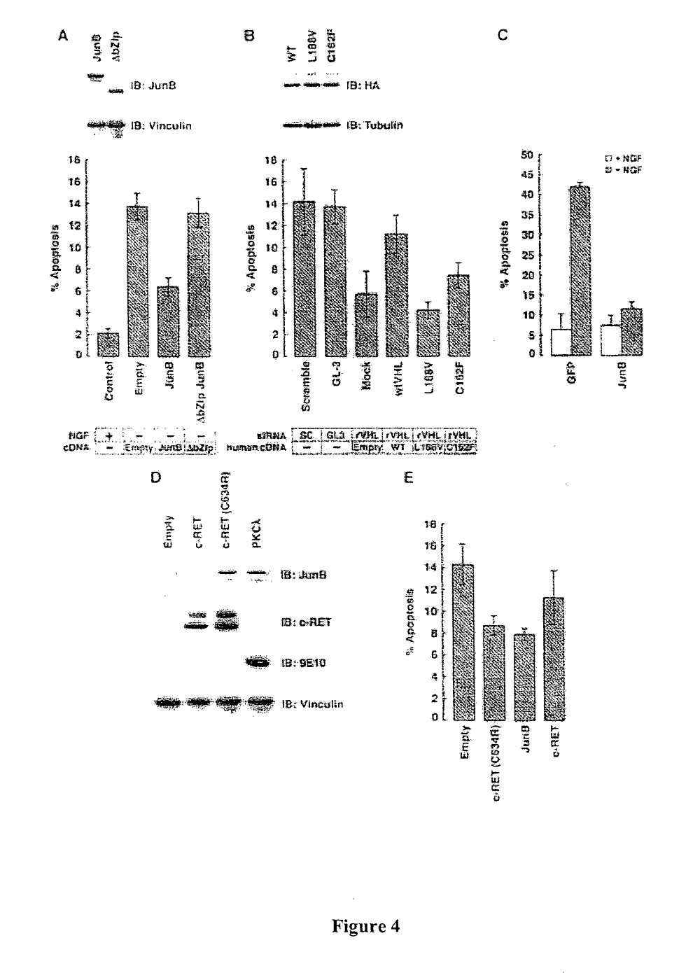

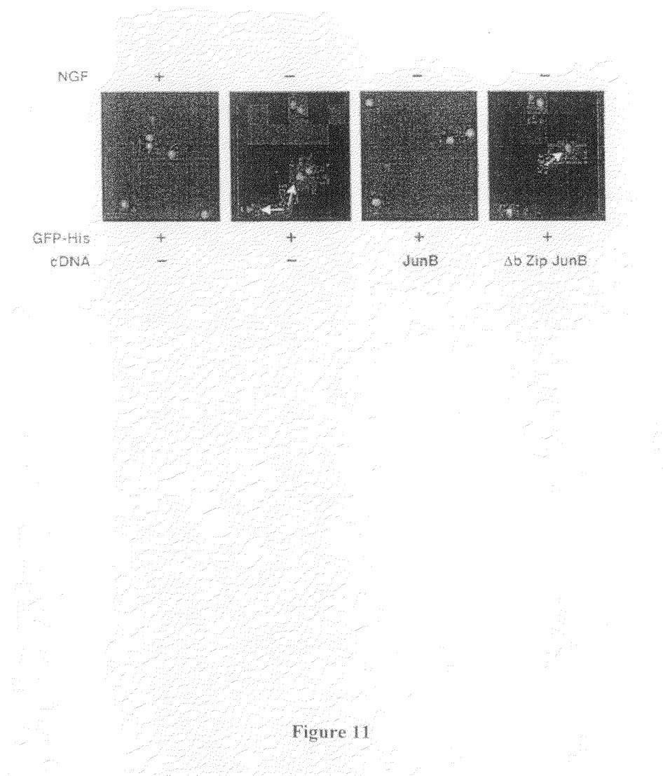

JunB was downregulated after NGF withdrawal from PC12 cells (FIG. 3C). Since JunB antagonizes c-Jun in many settings, we asked whether loss of pVHL or elevated JunB could block apoptosis after NGF withdrawal. In these experiments PC12 cells were again transfected with a plasmid encoding GFP-Histone to identify transfected cells and score apoptotic nuclei, in addition to the plasmid or siRNA of interest. After recovery from transfection the cells were grown in the presence of NGF for 5-7 days and then placed in NGF-free media. Apoptosis was substantially diminished by JunB (FIG. 4A and FIG. 11). This effect was specific because it was not observed with a dimerization-defective JunB mutant. Likewise, JunB suppressed apoptosis of rat primary sympathetic neurons after NGF deprivation (FIG. 4C). Treatment of PC12 with siRNA against rat VHL, but not various control siRNAs, also decreased apoptosis after NGF withdrawal (FIG. 4B). This effect was specifically due to downregulation of pVHL because it was reversed by a plasmid encoding wild-type human pVHL. Importantly, the best studied Type 2C pVHL mutant, L188V, did not reverse the effects of the VHL siRNA (FIG. 4B) despite its ability to downregulate HIF (FIG. 2C). In contrast, the elongin binding mutant pVHL C162F, which is grossly defective with respect to HIF regulation (Ohh et al. (2000) Nat Cell Biol 2:423-427) (see also FIG. 7E), was partially active in this assay. Collectively, these results implicate deregulation of JunB and escape from NGF-dependent apoptosis in the pathogenesis of VHL-associated pheochromocytoma. Similarly, an activated c-RET mutant linked to pheochromocytoma (C634R), but not wild-type c-RET, shown earlier to promote survival (De Vita et al. (2000) Cancer Res 60:3727-3731) induced JunB in PC12 cells and decreased apoptosis under NGF-poor conditions (FIGS. 4D and E).

### Example 4

### Induction of Neuronal Apoptosis is a Specific Attribute of EGLN3 and is Hydroxylase Dependent

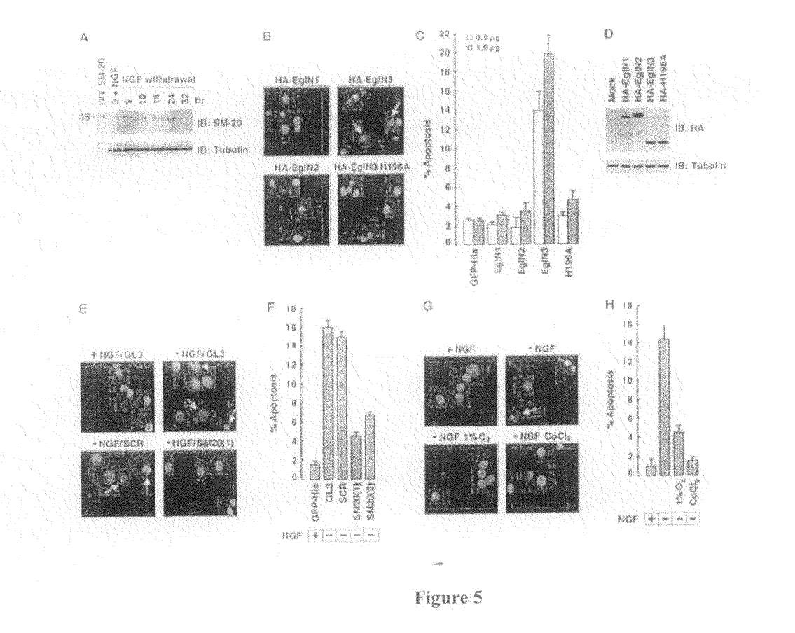

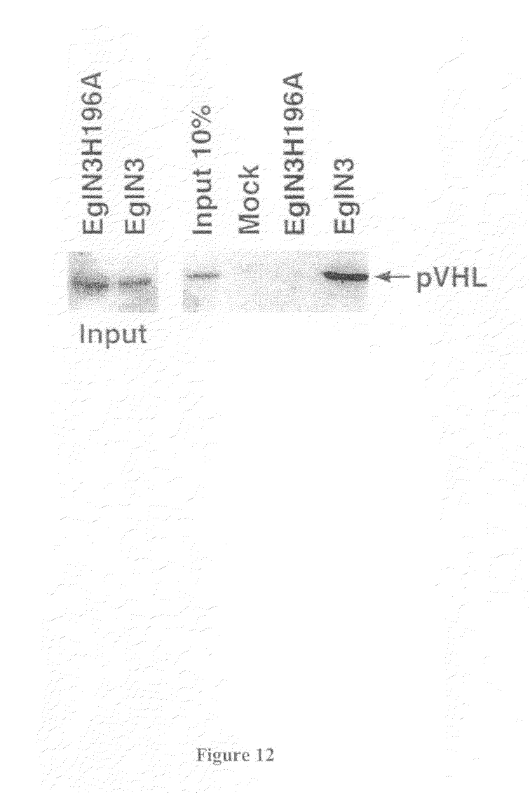

EGLN3, which in rat cells is called SM-20, was rapidly induced in PC12 cells after NGF withdrawal and killed these cells when ectopically expressed (Lipscomb et al. (1999) J Neurochem 73:429-432; Lipscomb et al. (2001) J Biol Chem 276:11775-11782; Straub et al. (2003) J Neurochem 85:318-328) (FIG. 5A and data not shown). We next transfected undifferentiated PC12 cells with plasmids encoding hemagglutinin (HA)-tagged versions of EGLN1, EGLN2, or EGLN3 along with the plasmid encoding GFP-histone. The nuclei of cells producing HA-EGLN1 or HA-EGLN2 appeared healthy 72 hours after transfection and were comparable to cells producing GFP-histone alone (FIGS. 5B and C). In contrast, the nuclei of 14-20% of the cells producing HA-EGLN3 displayed the nuclear hallmarks of apoptosis 48-72 hours after transfection. The fact that <20% of the cells appeared apoptotic at any point in time is reminiscent of the asynchronous cell death observed after NGF withdrawal. (Francois et al. (2001) Mol Cell Neurosci 18:347-362; Messam and Pittman (1998) Exp Cell Res 238:389-398.) Increased apoptosis was not observed in cells producing an EGLN3 variant in which a canonical histidine residue important for hydroxylase activity was converted to alanine (H196A) (FIGS. 5B and C and FIG. 12). Comparable amounts of the different EGLN species were produced in these experiments as determined by anti-HA immunoblot analysis (FIG. 5D). Therefore, induction of neuronal apoptosis is specific to EGLN3 among the EGLN family members and requires its enzymatic activity. C. elegans have a single EGLN gene called Eg1-9. (Epstein et al. (2001) Cell 107:43-54; Taylor (2001) Gene 275:125-132.) A role for EGLN in neuronal apoptosis is supported by the observation that Eg1-9 −/− worms are resistant to certain neurotoxins. (Darby et al. (1999) Proc Natl Acad Sci USA 96:15202-15207.)

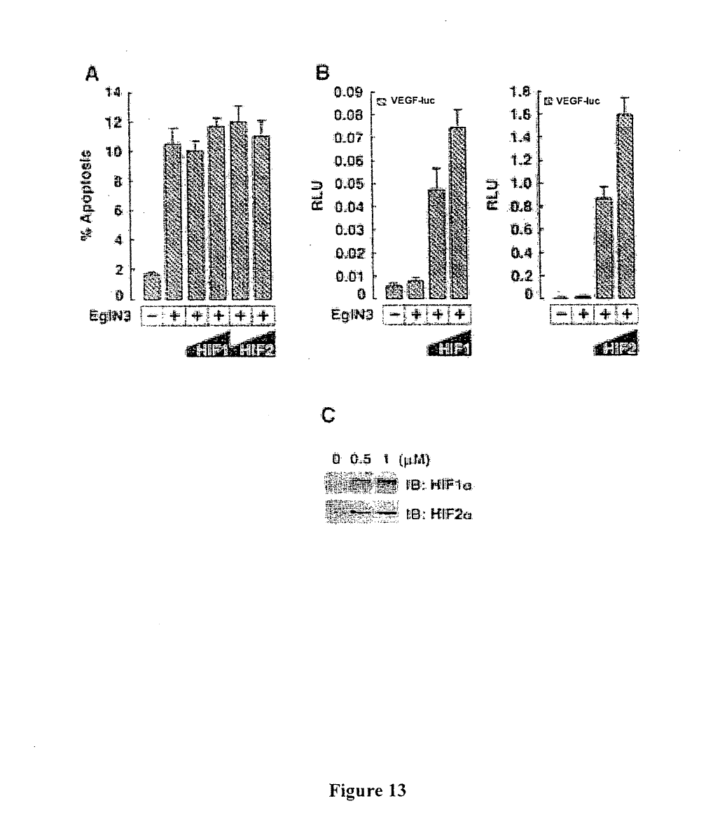

EGLN1, and not EGLN3, appears to be the primary HIF prolyl hydroxylase under normal conditions in cells. (Berra et al. (2003) EMBO J 22:4082-4090.) Moreover, EGLN3-induced apoptosis was not diminished when PC12 cells were cotransfected to produce HIF1α or HIF2α variants that can not be hydroxylated on proline (FIG. 13). Collectively, these results suggest that HIFα is not the relevant target of EGLN3 in this system.

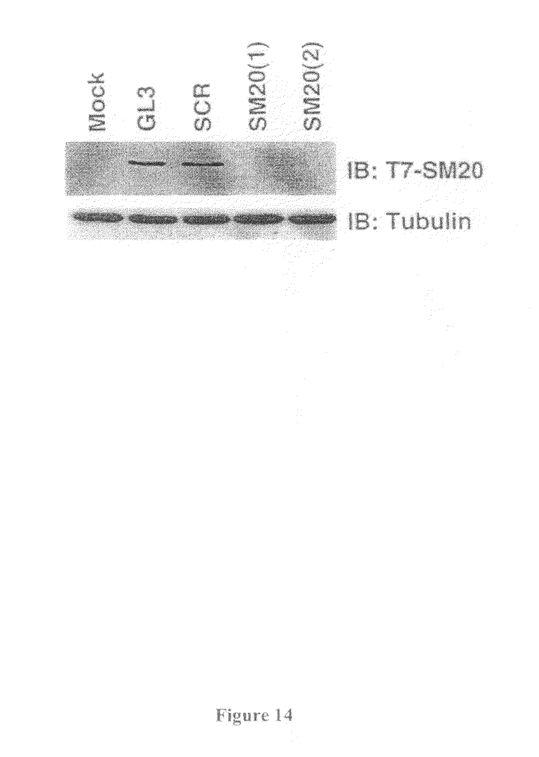

EGLN3/SM20-prolyl hydroxylase activity is required for apoptosis after NGF withdrawal. Transfection of PC12 cells with SM-20 siRNAs, but not various irrelevant or scrambled siRNAs, prior to differentiation and NGF withdrawal substantially decreased apoptosis (FIGS. 5E and F and FIG. 14), indicating that EGLN3/SM20 hydroxylase is necessary, as well as sufficient, for the induction of apoptosis by NGF withdrawal. Accordingly, apoptosis after NGF withdrawal was also decreased under low oxygen conditions or in the presence of cobalt chloride, both of which inhibit hydroxylase activity (FIGS. 5G and H).

### Example 5

### SDH Activity is Required for EGLN3/SM-20-Induced Neuronal Apoptosis

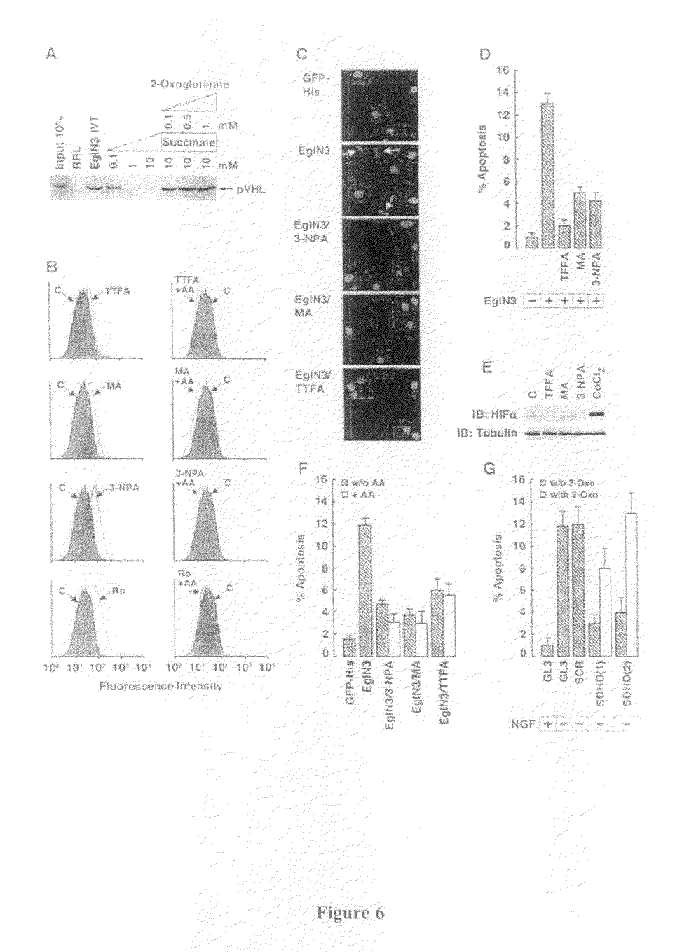

Prolyl hydroxylation by EGLN family members, which belong to a superfamily of 2-oxoglutarate-dependent dioxygenases, is coupled to conversion of 2-oxoglutarate (2-OG) into succinate. (Aravind and Koonin (2001) Genome Biol 2:RESEARCH0007; Gunzler and Weidmann (1998) In: Prolyl Hvdroxylase, Protein Disulfide Isomerase, and Other Structurally Related Proteins. (Guzman, Ed.) Marcel Dekker, Inc., New York N.Y., pp. 65-95; Schofield and Zhang (1999) Curr Opin Struct Biol 9:722-731.) SDH is an inner mitochondrial membrane enzyme that oxidizes succinate into fumarate as part of the Krebs cycle and also participates in electron transport. Two predictable outcomes of SDH inactivation would be the accumulation of succinate, which feedback inhibits 2-OG-dependent dioxygenases such as collagen prolyl hydroxylase and thymine-7-hydroxylase in vitro (Holme (1975) Biochemistry 14:4999-5003; Myllyla et al. (1977) Eur J Biochem 80:349-357), and increased production of reactive oxygen species (Lenaz et al. (2004) Biochim Biophys Acta 1658:89-94; McLennan and Degli Esposti (2000) J Bioenerg Biomembr 32-153-162; Yankovskaya et al. (2003) Science 299:700-704), which can inhibit EGLN activity (Gerald et al. (2004) Cell 118:781-794). To test whether succinate can also inhibit EGLN3 prolyl hydroxylase activity, we exploited the fact that EGLN3 can hydroxylate a HIF1α-derived peptide in vitro, as determined by capture of 35S-labeled pVHL. (Bruick and McKnight (2001) Science 294:1337-1340; Epstein et al. (2001) Cell 107:43-54.) As predicted, EGLN3 hydroxylase activity was diminished in the face of increasing amount of succinate (FIG. 5A). Hydroxylation activity was restored, however, by the addition of 2-OG (FIG. 6A), indicating that succinate and 2-OG act competitively in vitro. Intracellular succinate levels can approach 0.5 mM and are in vast excess of 2-OG following SDH inhibition. (Selak et al. (2005) Cancer Cell 7:77-85.) In addition, we confirmed that ROS production was increased in PC12 cells treated with pharmacological SDH inhibitors (FIG. 6B), consistent with findings obtained with cells expressing a mutated form of SDH C. (Ishii et al. (2005) Cancer Res 65:203-209.)

Motivated by these findings, we asked whether SDH activity influences EGLN3-induced apoptosis by cotransfecting undifferentiated PC12 cells with plasmids encoding HA-EGLN3 and GFP-Histone in the presence or absence of pharmacological SDH inhibitors. Three different inhibitors, malonic-Acid (MA), 3-nitroproprionic acid (3-NPA), and thenoyl trifluoroacetone (TTFA), conferred substantial protection against EGLN3-induced apoptosis (FIGS. 6C and D). Notably, HIFα protein levels were not increased by these agents, in contrast to PC12 cells treated with the hypoxia-mimetic cobalt chloride, which further argues that EGLN3-induced neuronal apoptosis is HIFα-independent (FIG. 6E). Coadministration of the antioxidant ascorbic acid failed to mitigate the effects of the SDH inhibitors on EGLN3-induced apoptosis despite blocking ROS induction (FIGS. 6B and F), suggesting that a non-ROS mechanism such as succinate accumulation attenuates EGLN3-induced apoptosis when SDH activity is impaired.

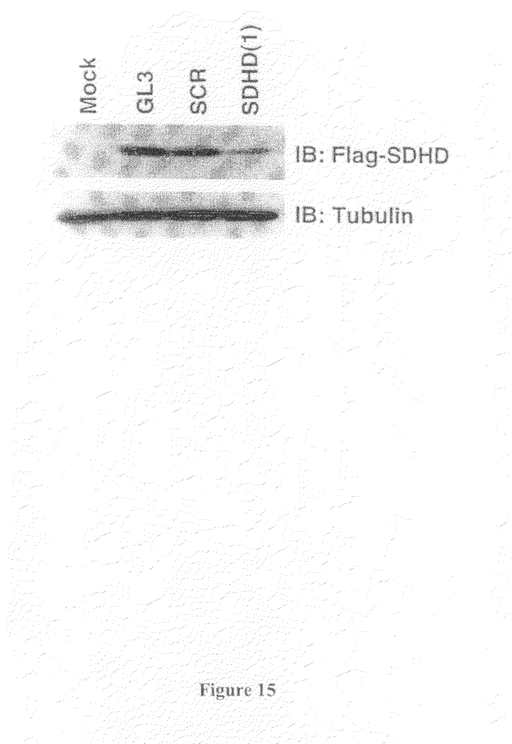

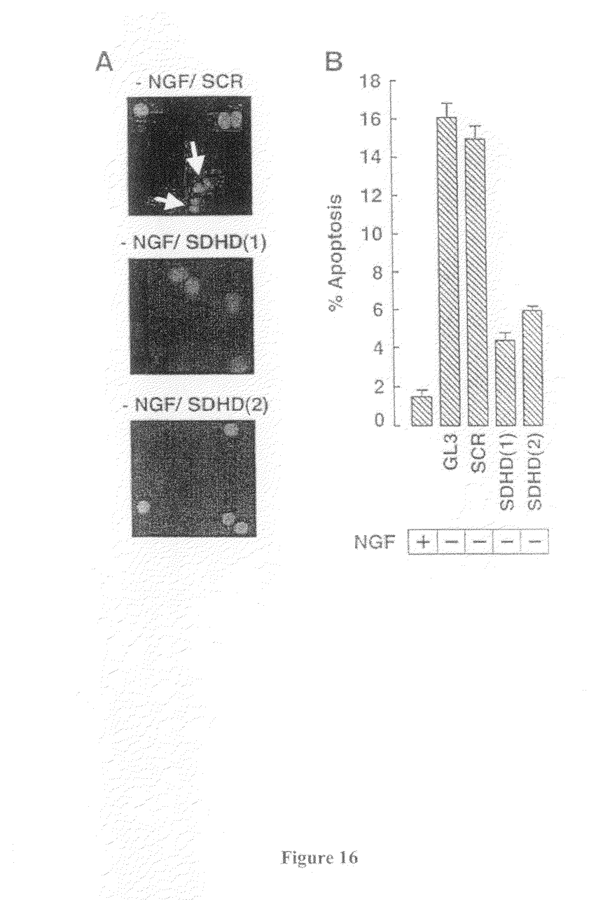

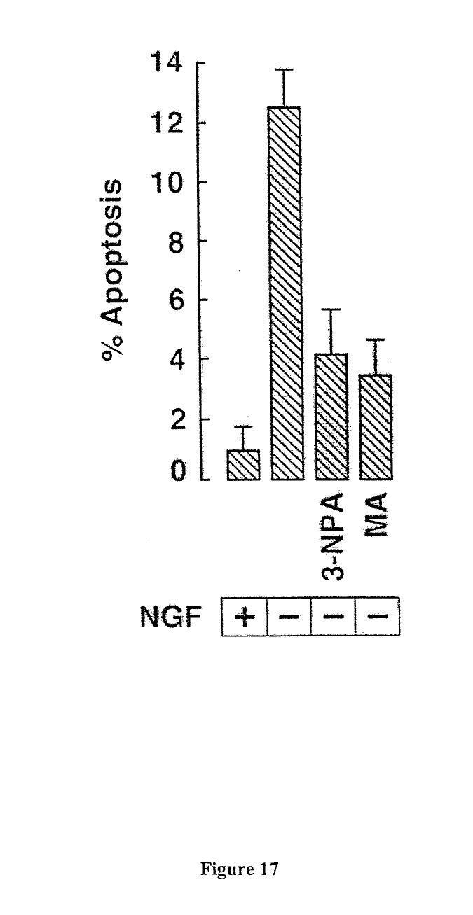

To assess the role of SDH in neuronal apoptosis in a more physiological context, we next transfected PC12 cells with the GFP-histone plasmid along with siRNAs prior to NGF treatment and withdrawal. Two different SDH D siRNAs, but not various control siRNAs, dramatically decreased apoptosis after NGF withdrawal (FIG. 6G and FIGS. 15 and 16). Notably, apoptosis was partially restored in this setting by the addition of 2-OG to the media (FIG. 6G). The SDH inhibitors (MA or 3-NPA) also diminished apoptosis in this setting (FIG. 17). Taken together, these findings suggest that inactivation of SDH, by promoting the accumulation of succinate, impairs EGLN3-induced apoptosis and promotes survival when NGF levels become limiting.

### Example 6

### c-Jun Acts Upstream of SM-20/EGLN3 in the NGF Signaling Pathway

We next asked if SM20/EGLN3 and c-Jun function in the same pathway. Apoptosis induced by overexpression of EGLN3 in PC12 cells, in contrast to that induced by NGF withdrawal, was not reduced by co-expression of the c-Jun antagonist JunB (FIG. 7A). In a reciprocal set of experiments, PC12 cells were cotransfected with plasmids encoding an activated, stabilized, version of c-Jun (Δc-Jun) and GFP-histone. Inclusion of SM20 siRNAs, but not control siRNAs, in the transfection mix dramatically reduced c-Jun-dependent apoptosis, arguing that SM-20/EGLN3 acts downstream or parallel of c-Jun (FIG. 7B). In support of the former possibility wild-type, but not mutant, c-Jun activated a luciferase reporter plasmid containing the SM-20 promoter in cotransfection assays (FIG. 7C) and SM-20 levels increased in PC12 cells infected with an adenovirus encoding c-Jun (FIG. 6D).

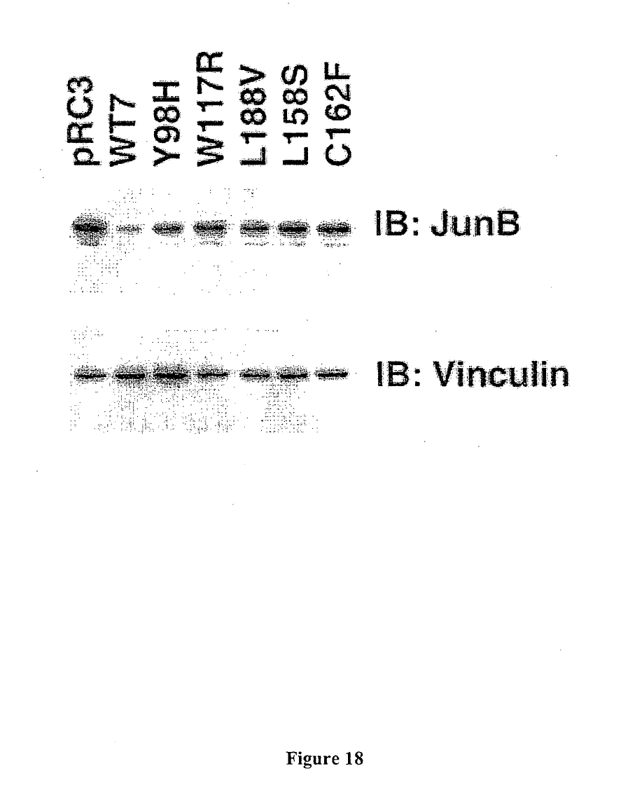

EGLN3 is induced by NGF withdrawal but also by HIF. (Aprelikova et al. (2004) J Cell Biochem 92:491-501; Cioffi et al. (2003) Biochem Biophys Res Commun 303:947-953; del Peso et al. (2003) J Biol Chem 278:48690-48695; Marxsen et al. (2004) Biochem J 381:761-767.) Therefore, pVHL has opposing effects on EGLN3 and the balance of those effects might dictate the risk of developing pheochromocytoma. In support of this, we detected high basal EGLN3 mRNA and protein levels in 786-O cells producing Type I pVHL mutants (as well as in 786-O cells stably transfected with an empty vector), which are associated with a low risk of pheochromocytoma, and low EGLN3 levels in 786-O cells producing Type 2 pVHL mutants, which are associated with a high risk of pheochromocytoma (FIGS. 7E and F). HIF and JunB levels in cells producing Type 2A and Type 2B pVHL mutants are comparable to those observed in cells producing Type I pVHL mutants (FIG. 7E and FIG. 18). Gene expression profiling indicates that a subset of HIF target genes, including EGLN3, are inhibited by Type 2 pVHL mutants despite increased HIF levels (data not shown).

### Example 7

### EGLN3/SM-20-Induced Neuronal Apoptosis

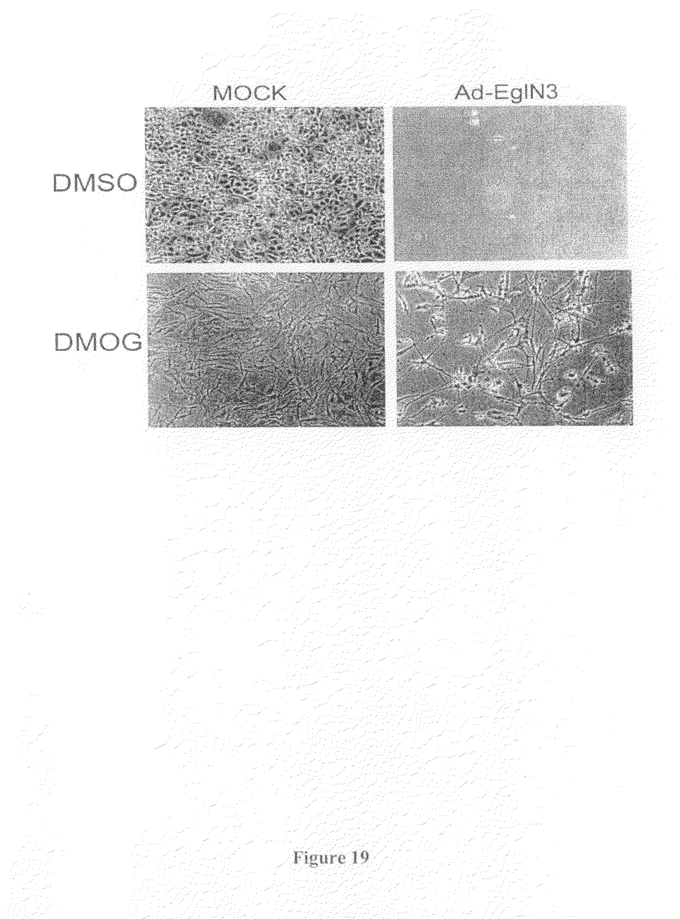

In experiments comparable to those described in Example 4, expression of EGLN3 induced apoptosis in a variety of murine and human cell lines of neural crest origin including cells derived from pheochromocytomas, neuroblastomas, and melanomas (see, e.g., FIG. 19). In contrast, EGLN3 did not induce apoptosis in a variety of epithelial cell lines.

A number of embodiments of the invention have been described. Nevertheless, it will be understood that various modifications may be made without departing from the spirit and scope of the invention. Accordingly, other embodiments are within the scope of the following claims.

1. A method for reducing apoptosis in a cell associated with or derived from the nervous system, the method comprising administering an inhibitor of EGLN3 enzyme activity to the cell.

2. The method of claim 1 wherein the cell is ex vivo.

3. The method of claim 1 wherein the cell is in vivo.

4. The method of claim 3 wherein the apoptosis is associated with a neurodegenerative disorder.

5. The method of claim 4 wherein the disorder is associated with the central nervous system.

6. The method of claim 4 wherein the disorder is associated with the peripheral nervous system.

7. The method of claim 4 wherein the disorder is selected from: Alzheimer's disease, Parkinson's disease, Huntington's disease, amyotrophic lateral sclerosis, multiple sclerosis, stroke, cerebral ischemia, AIDS-related dementia, neurodegeneration associated with bacterial infection, multi-infarct dementia, traumatic brain injury, spinal cord trauma, diabetic neuropathy, and neurodegeneration associated with aging.

8. The method of claim 1 wherein the inhibitor is a small molecule.

9. The method of claim 8 wherein the inhibitor is selected from the group consisting of malonic acid, 3-nitroproprionic acid, and theonyl trifluoracetone.

10. The method of claim 8 wherein the inhibitor is a 2-oxoglutarate analog.

11. The method of claim 10 wherein the analog is selected from the group consisting of dimethyloxalylglycine, N-oxalylglycine, N-oxalyl-2S-alanine, and N-oxalyl-2R-alanine.

12. A method comprising:
(a) identifying a patient suffering from or at risk for a neurodegenerative disorder; and
(b) administering to the identified patient an inhibitor of EGLN3 enzyme activity.

13. A method for increasing apoptosis in a subject having or at risk for having a cell proliferative disorder, the method comprising administering to the subject an agent that increases EGLN3 level or activity.

14. The method of claim 13, wherein the cell proliferative disorder is cancer.

15. The method of claim 14, wherein the cancer is derived from neural crest cells.

16. The method of claim 14, wherein the cancer is selected from the group consisting of a melanoma, neuroblastoma, small cell lung carcinoma, and pheochromocytoma.
